SEQUENCE LISTING (159 sequences)

SEQ ID NO: 1 - protein, 7 aa, Artificial Sequence
  CDR
ARGSVIL

SEQ ID NO: 2 - protein, 5 aa, Artificial Sequence
  CDR
DAWMD

SEQ ID NO: 3 - protein, 19 aa, Artificial Sequence
  CDR
EIRHKANDHAIFYXESVKG

SEQ ID NO: 4 - protein, 13 aa, Artificial Sequence
  CDR
TYYYGSSYGYCDV

SEQ ID NO: 5 - protein, 4 aa, Artificial Sequence
  CDR
PFAY

SEQ ID NO: 6 - protein, 11 aa, Artificial Sequence
  CDR
KASQSVGTTIV

SEQ ID NO: 7 - protein, 11 aa, Artificial Sequence
  CDR
RTSENIYSYLA

SEQ ID NO: 8 - protein, 7 aa, Artificial Sequence
  CDR
NAKTLAE

SEQ ID NO: 9 - protein, 7 aa, Artificial Sequence
  CDR
SASNRXT

SEQ ID NO: 10 - protein, 9 aa, Artificial Sequence
  CDR
QQYSSYPFT

SEQ ID NO: 11 - protein, 9 aa, Artificial Sequence
  CDR
QHHYGTPWT

SEQ ID NO: 12 - protein, 19 aa, Artificial Sequence
  CDR
EIRHKANDHAIFYAESVKG

SEQ ID NO: 13 - protein, 19 aa, Artificial Sequence
  CDR
EIRNKANNHARHYAESVKG

SEQ ID NO: 14 - protein, 19 aa, Artificial Sequence
  CDR
EIRHKANDYAIFYDESVKG

SEQ ID NO: 15 - protein, 19 aa, Artificial Sequence
  CDR
EIRHKANDHAIFYDESVKG

SEQ ID NO: 16 - protein, 19 aa, Artificial Sequence
  CDR
DIRNTANNHATFYAESVKG

SEQ ID NO: 17 - protein, 19 aa, Artificial Sequence
  CDR
EIRHKANDHAIFYDESVKG

SEQ ID NO: 18 - protein, 13 aa, Artificial Sequence
  CDR
TYYYGSSYGYCDV

SEQ ID NO: 19 - protein, 11 aa, Artificial Sequence
  CDR
KASQSVGTTIV

SEQ ID NO: 20 - protein, 11 aa, Artificial Sequence
  CDR
RTSENIYSYLA

SEQ ID NO: 21 - protein, 11 aa, Artificial Sequence
  CDR
KASQNVGTAVV

SEQ ID NO: 22 - protein, 7 aa, Artificial Sequence
  CDR
NAKTLAE

SEQ ID NO: 23 - protein, 7 aa, Artificial Sequence
  CDR
SASNRHT

SEQ ID NO: 24 - protein, 7 aa, Artificial Sequence
  CDR
SASTRYT

SEQ ID NO: 25 - protein, 7 aa, Artificial Sequence
  CDR
SASNRYT

SEQ ID NO: 26 - protein, 9 aa, Artificial Sequence
  CDR
QQYSSYPFT

SEQ ID NO: 27 - protein, 9 aa, Artificial Sequence
  CDR
QHHYGTPWT

SEQ ID NO: 28 - protein, 9 aa, Artificial Sequence
  CDR
QQYVNYPFT

SEQ ID NO: 29 - protein, 9 aa, Artificial Sequence
  CDR
QQYTSYPFT

SEQ ID NO: 30 - protein, 115 aa, Artificial Sequence
  Heavy chain
EVKLEESGGGLVQPGGSMKLSCAASGFTFSDAWMDWVRQSPEKGLEWVAEIRHKANDHAIFYXESVKGRFTISRDDSKSIVYLQMNSLRPEDTGIYYCTSPFAYWGQGTLVTVSA

SEQ ID NO: 31 - protein, 109 aa, Artificial Sequence
  Light chain
DIVMTQSQKFMSVTVGDRVSITCKASQSVGTTIVWYQQKPGQSPKLLIYSASNRXTGVPDRFTGSGSGTDFILTINNVQSEDLADYFCQQYSSYPFTFGSGTKLEIKRA

SEQ ID NO: 32 - protein, 115 aa, Artificial Sequence
  Heavy chain
EVKLEESGGGLVQPGGSMKLSCTASGFTFSDAWMDWVRQSPEKGLEWVAEIRHKANDHAIFYDESVKGRFTISRDDSKNIVYLQMNSLRPEDTGIYYCTSPFAYWGQGTLVTVSA

SEQ ID NO: 33 - protein, 115 aa, Artificial Sequence
  Heavy chain
EVKLEESGGGLGQPGGSMKLSCAASGFNFSDAWMDWVRQSPEKGLEWVAEIRHKANDHAIFYAESVKGRFTISRDDSKSSVYLQMNSLRAEDTGIYYCTSPFAYWGQGTLVTVSA

SEQ ID NO: 34 - protein, 115 aa, Artificial Sequence
  Heavy chain
EVKLEESGGGLVQPGGSMKLSCAASGITFSDAWMDWVRQSPEKGLEWVADIRNTANNHATFYAESVKGRFTISRDDSKSTVYLQMNTLRPEDTGIYYCTSPFAYWGQGTLVTVSA

SEQ ID NO: 35 - protein, 115 aa, Artificial Sequence
  Heavy chain
EVKFEESGGGLVQPGGSMKLSCAASGFIFSDAWMDWVRQSPEKGLEWVAEIRHKANDYAIFYDESVKGRFTISRDDSKSIVYLQMNNLRPEDTGIYYCTSPFAYWGQGTLVTVSA

SEQ ID NO: 36 - protein, 115 aa, Artificial Sequence
  Heavy chain
EVKLEESGGGLVQPGGSMKLSCAASGFTFSDAWMDWVRQSPEKGLEWVAEIRHKANDHAIFYDESVKGRFTISRDDSKSIVYLQMNSLRPEDTGIYYCTSPFAYWGQGTLVTVSA

SEQ ID NO: 37 - protein, 124 aa, Artificial Sequence
  Heavy chain
EVKLEESGGGLVQPGGSMKLSCAASGFTSSDAWMDWVRQSPEKGLEWVAEIRNKANNHARHYAESVKGRFTISRDDSKSSVYLQMNSLRAEDSGIYYCTRTYYYGSSYGYCDVWGTGTTVTVSS

SEQ ID NO: 38 - protein, 109 aa, Artificial Sequence
  Light chain
DIVMTQSQKFMSTTVGDRVSITCKASQNVGTAVVWFQQKPGQSPKLLIYSASNRYTRVPDRFTGSGSGTDFTLTISNVQSEDLADYFCQQYVNYPFTFGSGTKLEIKRA

SEQ ID NO: 39 - protein, 109 aa, Artificial Sequence
  Light chain
DIVMTQSQKFMSVTVGDRVSITCKASQSVGTTIVWYQQKPGQSPKLLIYSASNRHTGVPDRFTGSGSGTDFILTISNVQSEDLADYFCQQYSSYPFTFGSGTKLEIKRA

SEQ ID NO: 40 - protein, 109 aa, Artificial Sequence
  Light chain
DIVMTQSQRFMSVTVGDRVSITCKASQSVGTTIVWYQQKPGQSPKLLIYSASTRYTGVPDRFTGGGSGTDFILTINNVQSEDLADYFCQQYSSYPFTFGSGTKLEIKRA

SEQ ID NO: 41 - protein, 109 aa, Artificial Sequence
  Light chain
DIQMTQSPASLSASVGETVTITCRTSENIYSYLAWYQQKQGKSPQLLVYNAKTLAEGVPSRFSGSGSGTQFSLKINSLQPEDFGSYSCQHHYGTPWTFGGGTKLEIKRA

SEQ ID NO: 42 - protein, 109 aa, Artificial Sequence
  Light chain
DIVMTQSQKFMSVTVGDRVSITCKASQSVGTTIVWYQQKPGQSPKLLIYSASNRHTGVPDRFTGSGSGTDFILTINNVQSEDLADYFCQQYTSYPFTFGSGTKLEIKRA

SEQ ID NO: 43 - protein, 930 aa, Homo sapien
MLLGWASLLLCAFRLPLAAVGPAATPAQDKAGQPPTAAAAAQPRRRQGEEVQERAEPPGHPHPLAQRRRSKGLVQNIDQLYSGGGKVGYLVYAGGRRFLLDLERDGSVGIAGFVPAGGGTSAPWRHRSHCFYRGTVDGSPRSLAVFDLCGGLDGFFAVKHARYTLKPLLRGPWAEEEKGRVYGDGSARILHVYTREGFSFEALPPRASCETPASTPEAHEHAPAHSNPSGRAALASQLLDQSALSPAGGSGPQTWWRRRRRSISRARQVELLLVADASMARLYGRGLQHYLLTLASIANRLYSHASIENHIRLAVVKVVVLGDKDKSLEVSKNAATTLKNFCKWQHQHNQLGDDHEEHYDAAILFTREDLCGHHSCDTLGMADVGTICSPERSCAVIEDDGLHAAFTVAHEIGHLLGLSHDDSKFCEETFGSTEDKRLMSSILTSIDASKPWSKCTSATITEFLDDGHGNCLLDLPRKQILGPEELPGQTYDATQQCNLTFGPEYSVCPGMDVCARLWCAVVRQGQMVCLTKKLPAVEGTPCGKGRICLQGKCVDKTKKKYYSTSSHGNWGSWGSWGQCSRSCGGGVQFAYRHCNNPAPRNNGRYCTGKRAIYRSCSLMPCPPNGKSFRHEQCEAKNGYQSDAKGVKTFVEWVPKYAGVLPADVCKLTCRAKGTGYYVVFSPKVTDGTECRLYSNSVCVRGKCVRTGCDGIIGSKLQYDKCGVCGGDNSSCTKIVGTFNKKSKGYTDVVRIPEGATHIKVRQFKAKDQTRFTAYLALKKKNGEYLINGKYMISTSETIIDINGTVMNYSGWSHRDDFLHGMGYSATKEILIVQILATDPTKPLDVRYSFFVPKKSTPKVNSVTSHGSNKVGSHTSQPQWVTGPWLACSRTCDTGWHTRTVQCQDGNRKLAKGCPLSQRPSAFKQCLLKKC

SEQ ID NO: 44 - protein, 837 aa, Homo sapien [sequence omitted]

SEQ ID NO: 45 - DNA, 57 bp, Artificial Sequence
  Signal sequence
atgggctggtcctgcatcatcctgtttctggtggccaccgccaccggcgtgcacagc

SEQ ID NO: 46 - protein, 19 aa, Artificial Sequence
  Signal sequence
MGWSCIILFLVATATGVHS

SEQ ID NO: 47 - DNA, 372 bp, Murine
gaagtgaagcttgaggagtctggaggaggcttggtgcaacctggaggatccatgaaactctcttgtgctgcctctggattcacttctagtgacgcctggatggactgggtccgccagtctccagaaaaggggcttgagtgggttgctgaaattagaaacaaagctaataatcatgcaagacactatgctgagtctgtgaaagggaggttcaccatctcaagagatgattccaaaagtagtgtctacctgcaaatgaacagcttaagagctgaagactctggcatttattactgtaccaggacgtattattacggtagcagctacggatactgcgatgtctggggcacagggaccacggtcaccgtctcctca

SEQ ID NO: 48 - protein, 124 aa, Murine; identical to SEQ ID NO: 37

SEQ ID NO: 49 - DNA, 327 bp, Murine
gacatccagatgactcagtctccagcctccctatctgcatctgtgggagaaactgtcaccatcacatgtcgaacaagtgagaatatttacagttatttagcatggtatcagcagaaacagggaaaatctcctcagctcctggtctataatgcaaaaaccttagcagaaggtgtgccatcaaggttcagtggcagtggatcaggcacacagttttctctgaagatcaacagcctgcagcctgaagattttgggagttattcctgtcaacatcattatggtactccgtggacgttcggtggaggcaccaagctggaaatcaaacgggct

SEQ ID NO: 50 - protein, 109 aa, Murine; identical to SEQ ID NO: 41

SEQ ID NO: 51 - DNA, 345 bp, Murine
gaagtgaagcttgaggagtctggaggaggcttggtgcaacctggaggatccatgaaactctcctgtactgcctccggattcacttttagtgacgcctggatggactgggtccgccagtctccagagaagggacttgagtgggttgctgaaattagacacaaagctaatgatcatgcaatattttatgatgagtctgtgaaagggaggttcaccatctcaagagatgattccaaaaatattgtctatctgcaaatgaacagtttaagacctgaggacaccggcatttattattgtaccagtccttttgcttattggggccaagggactctggtcactgtctctgca

SEQ ID NO: 52 - protein, 115 aa, Murine; identical to SEQ ID NO: 32

SEQ ID NO: 53 - DNA, 327 bp, Murine
gacattgtgatgacccagtctcaaaaattcatgtccgtaacagtgggagacagggtcagcatcacctgcaaggccagtcagagtgtgggaactactatagtctggtatcaacagaaaccaggacaatctcctaaattactgatttactctgcatccaatcggcacactggggtccctgatcgcttcacaggcagtggatctgggacagatttcattctcaccattaacaatgtgcagtctgaggacctggcagattatttctgtcagcaatatacctcctatccattcacgttcggctcggggacaaagttggagataaaacgggct

SEQ ID NO: 54 - protein, 109 aa, Murine; identical to SEQ ID NO: 42

SEQ ID NO: 55 - protein, 5 aa, Artificial Sequence
  CDR
DAWMD

SEQ ID NO: 56 - protein, 19 aa, Artificial Sequence
  CDR
EIRNKANNHARHYAESVKG

SEQ ID NO: 57 - protein, 13 aa, Artificial Sequence
  CDR
TYYYGSSYGYCDV

SEQ ID NO: 58 - protein, 11 aa, Artificial Sequence
  CDR
RTSENIYSYLA

SEQ ID NO: 59 - protein, 7 aa, Artificial Sequence
  CDR
NAKTLAE

SEQ ID NO: 60 - protein, 9 aa, Artificial Sequence
  CDR
QHHYGTPWT

SEQ ID NO: 61 - protein, 5 aa, Artificial Sequence
  CDR
DAWMD

SEQ ID NO: 62 - protein, 19 aa, Artificial Sequence
  CDR
EIRHKANDHAIFYDESVKG

SEQ ID NO: 63 - protein, 4 aa, Artificial Sequence
  CDR
PFAY

SEQ ID NO: 64 - protein, 11 aa, Artificial Sequence
  CDR
KASQSVGTTIV

SEQ ID NO: 65 - protein, 7 aa, Artificial Sequence
  CDR
SASNRHT

SEQ ID NO: 66 - protein, 9 aa, Artificial Sequence
  CDR
QQYTSYPFT

SEQ ID NO: 67 - DNA, 1362 bp, Artificial Sequence
  Heavy chain
gaggtgaagctggaggaatcaggaggcggactggtgcagcccggcggcagcatgaaactgagctgcgctgcctctggcttcaccagctctgacgcctggatggattgggtgaggcagagccccgagaagggcctggagtgggtggccgagatcaggaacaaggccaacaaccacgccaggcactacgccgagagcgtgaagggcaggttcaccatcagcagggacgacagcaagagctccgtgtacctgcagatgaacagcctccgggccgaggacagcggcatctactactgcaccaggacctactactacgggagcagctacggctattgcgacgtgtggggaaccggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 68 - protein, 454 aa, Artificial Sequence
  Heavy chain
EVKLEESGGGLVQPGGSMKLSCAASGFTSSDAWMDWVRQSPEKGLEWVAEIRNKANNHARHYAESVKGRFTISRDDSKSSVYLQMNSLRAEDSGIYYCTRTYYYGSSYGYCDVWGTGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - DNA, 642 bp, Artificial Sequence
  Light chain
gacatccagatgacccagagccccgccagcctgagcgcaagcgtgggcgagaccgtcaccatcacctgcaggaccagcgagaacatctacagctacctggcctggtatcagcagaagcagggcaagagcccccagctgctggtgtacaacgccaaaaccctggccgaaggcgtgccctctaggttcagcggaagcggaagcggcacccagttcagcctcaagatcaactccctgcagcccgaggacttcggcagctacagctgccagcaccactacggcactccctggacctttggcggcggcaccaagctggagattaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 70 - protein, 214 aa, Artificial Sequence
  Light chain
DIQMTQSPASLSASVGETVTITCRTSENIYSYLAWYQQKQGKSPQLLVYNAKTLAEGVPSRFSGSGSGTQFSLKINSLQPEDFGSYSCQHHYGTPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - DNA, 1335 bp, Artificial Sequence
  Heavy chain
gaggtgaaactggaggaaagcggcggcggactggtgcagcccggcggctcaatgaagctgagctgcaccgccagcggcttcaccttctctgacgcctggatggactgggtgaggcagagccccgagaagggcctggagtgggtggctgagatcaggcacaaggccaacgaccacgccatcttctacgacgagagcgtgaagggcaggttcaccatcagcagggacgacagcaagaacatcgtgtacctccagatgaacagcctgaggcccgaggataccggcatctactactgcaccagcccctttgcctattggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 72 - protein, 445 aa, Artificial Sequence
  Heavy chain
EVKLEESGGGLVQPGGSMKLSCTASGFTFSDAWMDWVRQSPEKGLEWVAEIRHKANDHAIFYDESVKGRFTISRDDSKNIVYLQMNSLRPEDTGIYYCTSPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - DNA, 642 bp, Artificial Sequence
  Light chain
gacatcgtgatgacccagagccagaaattcatgagcgtgaccgtgggcgacagggtgtccatcacttgcaaggccagccagagcgtgggcaccactatcgtgtggtaccagcagaagcccggccagagccccaagctcctgatctacagcgccagcaacaggcacaccggagtgcccgacaggtttactggcagcggctcaggcaccgacttcatcctgaccatcaacaacgtgcagagcgaggacctggccgattacttctgccagcagtacaccagctaccccttcaccttcggctcagggaccaagctggagattaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 74 - protein, 214 aa, Artificial Sequence
  Light chain
DIVMTQSQKFMSVTVGDRVSITCKASQSVGTTIVWYQQKPGQSPKLLIYSASNRHTGVPDRFTGSGSGTDFILTINNVQSEDLADYFCQQYTSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - DNA, 372 bp, Artificial Sequence
  Heavy chain
gaggtgaagctggaggaatcaggaggcggactggtgcagcccggcggcagcatgaaactgagctgcgctgcctctggcttcaccagctctgacgcctggatggattgggtgaggcagagccccgagaagggcctggagtgggtggccgagatcaggaacaaggccaacaaccacgccaggcactacgccgagagcgtgaagggcaggttcaccatcagcagggacgacagcaagagctccgtgtacctgcagatgaacagcctccgggccgaggacagcggcatctactactgcaccaggacctactactacgggagcagctacggctattgcgacgtgtggggaaccggcacactagtcaccgtgagcagc

SEQ ID NO: 76 - protein, 124 aa, Artificial Sequence
  Heavy chain
EVKLEESGGGLVQPGGSMKLSCAASGFTSSDAWMDWVRQSPEKGLEWVAEIRNKANNHARHYAESVKGRFTISRDDSKSSVYLQMNSLRAEDSGIYYCTRTYYYGSSYGYCDVWGTGTLVTVSS

SEQ ID NO: 77 - DNA, 327 bp, Artificial Sequence
  Light chain
gacatccagatgacccagagccccgccagcctgagcgcaagcgtgggcgagaccgtcaccatcacctgcaggaccagcgagaacatctacagctacctggcctggtatcagcagaagcagggcaagagcccccagctgctggtgtacaacgccaaaaccctggccgaaggcgtgccctctaggttcagcggaagcggaagcggcacccagttcagcctcaagatcaactccctgcagcccgaggacttcggcagctacagctgccagcaccactacggcactccctggacctttggcggcggcaccaagctggagattaagcgtacg

SEQ ID NO: 78 - protein, 109 aa, Artificial Sequence
  Light chain
DIQMTQSPASLSASVGETVTITCRTSENIYSYLAWYQQKQGKSPQLLVYNAKTLAEGVPSRFSGSGSGTQFSLKINSLQPEDFGSYSCQHHYGTPWTFGGGTKLEIKRT

SEQ ID NO: 79 - DNA, 345 bp, Artificial Sequence
  Heavy chain
gaggtgaaactggaggaaagcggcggcggactggtgcagcccggcggctcaatgaagctgagctgcaccgccagcggcttcaccttctctgacgcctggatggactgggtgaggcagagccccgagaagggcctggagtgggtggctgagatcaggcacaaggccaacgaccacgccatcttctacgacgagagcgtgaagggcaggttcaccatcagcagggacgacagcaagaacatcgtgtacctccagatgaacagcctgaggcccgaggataccggcatctactactgcaccagcccctttgcctattggggccagggcacactagtcaccgtgtccagc

SEQ ID NO: 80 - protein, 115 aa, Artificial Sequence
  Heavy chain
EVKLEESGGGLVQPGGSMKLSCTASGFTFSDAWMDWVRQSPEKGLEWVAEIRHKANDHAIFYDESVKGRFTISRDDSKNIVYLQMNSLRPEDTGIYYCTSPFAYWGQGTLVTVSS

SEQ ID NO: 81 - DNA, 327 bp, Artificial Sequence
  Light chain
gacatcgtgatgacccagagccagaaattcatgagcgtgaccgtgggcgacagggtgtccatcacttgcaaggccagccagagcgtgggcaccactatcgtgtggtaccagcagaagcccggccagagccccaagctcctgatctacagcgccagcaacaggcacaccggagtgcccgacaggtttactggcagcggctcaggcaccgacttcatcctgaccatcaacaacgtgcagagcgaggacctggccgattacttctgccagcagtacaccagctaccccttcaccttcggctcagggaccaagctggagattaagcgtacg

SEQ ID NO: 82 - protein, 109 aa, Artificial Sequence
  Light chain
DIVMTQSQKFMSVTVGDRVSITCKASQSVGTTIVWYQQKPGQSPKLLIYSASNRHTGVPDRFTGSGSGTDFILTINNVQSEDLADYFCQQYTSYPFTFGSGTKLEIKRT

SEQ ID NO: 83 - DNA, 1362 bp, Artificial Sequence
  Heavy chain
gaggtgcagctggtcgaaagcggcggcggcctggtgcagcccggaggcagcctcaggctgtcttgcgctgcctccggcttcaccttcagcgacgcctggatggattgggtgaggcaggcccccggcaaaggcctggagtgggtggccgagatcaggaacaaggccaacaaccacgccaggcactacgccgagagcgtgaaggggaggttcaccatcagcagggacaacgccaagaacagcctgtacctgcagatgaacagcctgagggccgaggacaccgccgtgtactactgcgcccggacctactactacgggagcagctacggctattgcgacgtgtggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 84 - protein, 454 aa, Artificial Sequence
  Heavy chain
EVQLVESGGGLVQPGGSLRLSCAASGFTFSDAWMDWVRQAPGKGLEWVAEIRNKANNHARHYAESVKGRFTISRDNAKNSLYLQMNSLRAEDTAVYYCARTYYYGSSYGYCDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - DNA, 1362 bp, Artificial Sequence
  Heavy chain
gaggtgcagctggtcgaaagcggcggcggcctggtgcagcccggaggcagcctcaggctgtcttgcgctgcctccggcttcaccttcagcgacgcctggatggattgggtgaggcaggcccccggcaaaggcctggagtgggtggccgagatcaggaacaaggccaacaaccacgccaggcactacgccgagagcgtgaaggggaggttcaccatcagcagggacaacgccaagaacagcctgtacctgcagatgaacagcctgagggccgaggacaccgccgtgtactactgcacccggacctactactacgggagcagctacggctattgcgacgtgtggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 86 - protein, 454 aa, Artificial Sequence
  Heavy chain
EVQLVESGGGLVQPGGSLRLSCAASGFTFSDAWMDWVRQAPGKGLEWVAEIRNKANNHARHYAESVKGRFTISRDNAKNSLYLQMNSLRAEDTAVYYCTRTYYYGSSYGYCDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - DNA, 1362 bp, Artificial Sequence
  Heavy chain
gaggtgcagctggtcgaaagcggcggcggcctggtgcagcccggaggcagcctcaggctgtcttgcgctgcctccggcttcaccagcagcgacgcctggatggattgggtgaggcaggcccccggcaaaggcctggagtgggtggccgagatcaggaacaaggccaacaaccacgccaggcactacgccgagagcgtgaaggggaggttcaccatcagcagggacaacgccaagaacagcctgtacctgcagatgaacagcctgagggccgaggacaccgccgtgtactactgcgcccggacctactactacgggagcagctacggctattgcgacgtgtggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 88 - protein, 454 aa, Artificial Sequence
  Heavy chain
EVQLVESGGGLVQPGGSLRLSCAASGFTSSDAWMDWVRQAPGKGLEWVAEIRNKANNHARHYAESVKGRFTISRDNAKNSLYLQMNSLRAEDTAVYYCARTYYYGSSYGYCDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - DNA, 1362 bp, Artificial Sequence
  Heavy chain
gaggtgcagctggtcgaaagcggcggcggcctggtgcagcccggaggcagcctcaggctgtcttgcgctgcctccggcttcaccagcagcgacgcctggatggattgggtgaggcaggcccccggcaaaggcctggagtgggtggccgagatcaggaacaaggccaacaaccacgccaggcactacgccgagagcgtgaaggggaggttcaccatcagcagggacaacgccaagaacagcctgtacctgcagatgaacagcctgagggccgaggacaccgccgtgtactactgcacccggacctactactacgggagcagctacggctattgcgacgtgtggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 90 - protein, 454 aa, Artificial Sequence
  Heavy chain
EVQLVESGGGLVQPGGSLRLSCAASGFTSSDAWMDWVRQAPGKGLEWVAEIRNKANNHARHYAESVKGRFTISRDNAKNSLYLQMNSLRAEDTAVYYCTRTYYYGSSYGYCDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - DNA, 1362 bp, Artificial Sequence
  Heavy chain
gaggtgcagctggtcgaaagcggcggcggcctggtgcagcccggaggcagcctcaggctgtcttgcgctgcctccggcttcaccagcagcgacgcctggatggattgggtgaggcaggcccccggcaaaggcctggagtgggtggccgagatcaggaacaaggccaacaaccacgccaggcactacgccgagagcgtgaaggggaggttcaccatcagcagggacgacgccaagaacagcctgtacctgcagatgaacagcctgagggccgaggacaccgccgtgtactactgcacccggacctactactacgggagcagctacggctattgcgacgtgtggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 92 - protein, 454 aa, Artificial Sequence
  Heavy chain
EVQLVESGGGLVQPGGSLRLSCAASGFTSSDAWMDWVRQAPGKGLEWVAEIRNKANNHARHYAESVKGRFTISRDDAKNSLYLQMNSLRAEDTAVYYCTRTYYYGSSYGYCDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - DNA, 1362 bp, Artificial Sequence
  Heavy chain
gaggtgcagctggtcgaaagcggcggcggcctggtgcagcccggaggcagcctcaggctgtcttgcgctgcctccggcttcaccagcagcgacgcctggatggattgggtgaggcaggcccccggcaaaggcctggagtgggtggccgagatcaggaacaaggccaacaaccacgccaggcactacgccgagagcgtgaaggggaggttcaccatcagcagggacaacgccaagaacagcgtctacctgcagatgaacagcctgagggccgaggacaccgccgtgtactactgcacccggacctactactacgggagcagctacggctattgcgacgtgtggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 94 - protein, 454 aa, Artificial Sequence
  Heavy chain
EVQLVESGGGLVQPGGSLRLSCAASGFTSSDAWMDWVRQAPGKGLEWVAEIRNKANNHARHYAESVKGRFTISRDNAKNSVYLQMNSLRAEDTAVYYCTRTYYYGSSYGYCDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - DNA, 1362 bp, Artificial Sequence
  Heavy chain
gaggtgcagctggtcgaaagcggcggcggcctggtgcagcccggaggcagcctcaggctgtcttgcgctgcctccggcttcaccagcagcgacgcctggatggattgggtgaggcaggcccccggcaaaggcctggagtgggtggccgagatcaggaacaaggccaacaaccacgccaggcactacgccgagagcgtgaaggggaggttcaccatcagcagggacgacgccaagaacagcgtctacctgcagatgaacagcctgagggccgaggacaccgccgtgtactactgcacccggacctactactacgggagcagctacggctattgcgacgtgtggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 96 - protein, 454 aa, Artificial Sequence
  Heavy chain
EVQLVESGGGLVQPGGSLRLSCAASGFTSSDAWMDWVRQAPGKGLEWVAEIRNKANNHARHYAESVKGRFTISRDDAKNSVYLQMNSLRAEDTAVYYCTRTYYYGSSYGYCDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - DNA, 642 bp, Artificial Sequence
  Light chain
gacatccagatgacccagagccccagcagcctgagcgctagcgtgggcgacagggtgaccatcacctgcaggacctccgagaacatctacagctacctggcctggtatcagcagaaacccggcaaggcccccaagctcctgatctacaacgccaagacactggccgagggcgtgcccagcaggttctcaggcagcggctctgggaccgacttcaccctgaccatcagcagcctgcagcccgaggacttcgccacctactactgccagcaccactacggaaccccctggaccttcggccagggcaccaagctggagattaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 98 - protein, 214 aa, Artificial Sequence
  Light chain
DIQMTQSPSSLSASVGDRVTITCRTSENIYSYLAWYQQKPGKAPKLLIYNAKTLAEGVPSRFSGSGSGTDFTLTISSLQPEDFATYYCQHHYGTPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - DNA, 642 bp, Artificial Sequence
  Light chain
gacatccagatgacccagagccccagcagcctgagcgctagcgtgggcgacagggtgaccatcacctgcaggacctccgagaacatctacagctacctggcctggtatcagcagaaacccggcaaggcccccaagctcctggtgtacaacgccaagacactggccgagggcgtgcccagcaggttctcaggcagcggctctgggaccgacttcaccctgaccatcagcagcctgcagcccgaggacttcgccacctactactgccagcaccactacggaaccccctggaccttcggccagggcaccaagctggagattaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 100 - protein, 214 aa, Artificial Sequence
  Light chain
DIQMTQSPSSLSASVGDRVTITCRTSENIYSYLAWYQQKPGKAPKLLVYNAKTLAEGVPSRFSGSGSGTDFTLTISSLQPEDFATYYCQHHYGTPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - DNA, 642 bp, Artificial Sequence
  Light chain
gacatccagatgacccagagccccagcagcctgagcgctagcgtgggcgacagggtgaccatcacctgcaggacctccgagaacatctacagctacctggcctggtatcagcagaaacccggcaaggccccccagctcctggtgtacaacgccaagacactggccgagggcgtgcccagcaggttctcaggcagcggctctgggaccgacttcaccctgaccatcagcagcctgcagcccgaggacttcgccacctactactgccagcaccactacggaaccccctggaccttcggccagggcaccaagctggagattaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 102 - protein, 214 aa, Artificial Sequence
  Light chain
DIQMTQSPSSLSASVGDRVTITCRTSENIYSYLAWYQQKPGKAPQLLVYNAKTLAEGVPSRFSGSGSGTDFTLTISSLQPEDFATYYCQHHYGTPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - DNA, 1335 bp, Artificial Sequence
  Heavy chain
gaggtgcagctcctggagagcggcgggggcctggtgcagcccggcggcagcctgagactgagctgcgccgcaagcggcttcaccttcagcgacgcctggatggattgggtgaggcaggcccccggcaaaggactggagtgggtcagcgagatcaggcacaaggccaacgaccacgccatcttctacgacgagagcgtgaagggcaggttcaccatctccagggacaacagcaagaacaccctgtacctgcagatgaacagcctgagggccgaagacaccgctgtgtactactgcgccaagccctttgcctactggggccagggaacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 104 - protein, 445 aa, Artificial Sequence
  Heavy chain
EVQLLESGGGLVQPGGSLRLSCAASGFTFSDAWMDWVRQAPGKGLEWVSEIRHKANDHAIFYDESVKGRFTISRDNSKNTLYLQMNSLRAEDTAVYYCAKPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - DNA, 1335 bp, Artificial Sequence
  Heavy chain
gaggtgcagctcctggagagcggcgggggcctggtgcagcccggcggcagcctgagactgagctgcgccgcaagcggcttcaccttcagcgacgcctggatggattgggtgaggcaggcccccggcaaaggactggagtgggtcagcgagatcaggcacaaggccaacgaccacgccatcttctacgacgagagcgtgaagggcaggttcaccatctccagggacaacagcaagaacaccctgtacctgcagatgaacagcctgagggccgaagacaccgctgtgtactactgcaccagcccctttgcctactggggccagggaacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 106 - protein, 445 aa, Artificial Sequence
  Heavy chain
EVQLLESGGGLVQPGGSLRLSCAASGFTFSDAWMDWVRQAPGKGLEWVSEIRHKANDHAIFYDESVKGRFTISRDNSKNTLYLQMNSLRAEDTAVYYCTSPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - DNA, 1335 bp, Artificial Sequence
  Heavy chain
gaggtgcagctcctggagagcggcgggggcctggtgcagcccggcggcagcctgagactgagctgcgccgcaagcggcttcaccttcagcgacgcctggatggattgggtgaggcaggcccccggcaaaggactggagtgggtcagcgagatcaggcacaaggccaacgaccacgccatcttctacgacgagagcgtgaagggcaggttcaccatctccagggacgacagcaagaacaccctgtacctgcagatgaacagcctgagggccgaagacaccgctgtgtactactgcaccagcccctttgcctactggggccagggaacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 108 - protein, 445 aa, Artificial Sequence
  Heavy chain
EVQLLESGGGLVQPGGSLRLSCAASGFTFSDAWMDWVRQAPGKGLEWVSEIRHKANDHAIFYDESVKGRFTISRDDSKNTLYLQMNSLRAEDTAVYYCTSPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - DNA, 1335 bp, Artificial Sequence
  Heavy chain
gaggtgcagctcctggagagcggcgggggcctggtgcagcccggcggcagcctgagactgagctgcgccgcaagcggcttcaccttcagcgacgcctggatggattgggtgaggcaggcccccggcaaaggactggagtgggtcgccgagatcaggcacaaggccaacgaccacgccatcttctacgacgagagcgtgaagggcaggttcaccatctccagggacaacagcaagaacaccgtgtacctgcagatgaacagcctgagggccgaagacaccgctgtgtactactgcaccagcccctttgcctactggggccagggaacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 110 - protein, 445 aa, Artificial Sequence
  Heavy chain
EVQLLESGGGLVQPGGSLRLSCAASGFTFSDAWMDWVRQAPGKGLEWVAEIRHKANDHAIFYDESVKGRFTISRDNSKNTVYLQMNSLRAEDTAVYYCTSPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - DNA, 1335 bp, Artificial Sequence
  Heavy chain
gaggtgcagctcctggagagcggcgggggcctggtgcagcccggcggcagcctgagactgagctgcgccgcaagcggcttcaccttcagcgacgcctggatggattgggtgaggcaggcccccggcaaaggactggagtgggtcgccgagatcaggcacaaggccaacgaccacgccatcttctacgacgagagcgtgaagggcaggttcaccatctccagggacgacagcaagaacaccgtgtacctgcagatgaacagcctgagggccgaagacaccgctgtgtactactgcaccagcccctttgcctactggggccagggaacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 112 - protein, 445 aa, Artificial Sequence
  Heavy chain
EVQLLESGGGLVQPGGSLRLSCAASGFTFSDAWMDWVRQAPGKGLEWVAEIRHKANDHAIFYDESVKGRFTISRDDSKNTVYLQMNSLRAEDTAVYYCTSPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - DNA, 642 bp, Artificial Sequence
  Light chain
gacatccagatgacccagagcccctcaagcctgtctgccagcgtgggcgacagggtgaccattacctgcaaggccagccagagcgtcggcaccaccatcgtgtggtaccagcagaaacccggcaaggcccccaagctcctgatctacagcgccagcaacaggcacaccggcgtgcctagcaggtttagcggaagcggcagcggcaccgacttcaccctgacaatcagcagcctgcagcccgaggacttcgccacctactactgccagcagtacaccagctatcccttcaccttcggccagggcaccaagctggagatcaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 114 - protein, 214 aa, Artificial Sequence
  Light chain
DIQMTQSPSSLSASVGDRVTITCKASQSVGTTIVWYQQKPGKAPKLLIYSASNRHTGVPSRFSGSGSGTDFTLTISSLQPEDFATYYCQQYTSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - DNA, 372 bp, Artificial Sequence
  Heavy chain
gaggtgcagctggtcgaaagcggcggcggcctggtgcagcccggaggcagcctcaggctgtcttgcgctgcctccggcttcaccttcagcgacgcctggatggattgggtgaggcaggcccccggcaaaggcctggagtgggtggccgagatcaggaacaaggccaacaaccacgccaggcactacgccgagagcgtgaaggggaggttcaccatcagcagggacaacgccaagaacagcctgtacctgcagatgaacagcctgagggccgaggacaccgccgtgtactactgcgcccggacctactactacgggagcagctacggctattgcgacgtgtggggccagggcacactagtgaccgtgtccagc

SEQ ID NO: 116 - protein, 124 aa, Artificial Sequence
  Heavy chain
EVQLVESGGGLVQPGGSLRLSCAASGFTFSDAWMDWVRQAPGKGLEWVAEIRNKANNHARHYAESVKGRFTISRDNAKNSLYLQMNSLRAEDTAVYYCARTYYYGSSYGYCDVWGQGTLVTVSS

SEQ ID NO: 117 - DNA, 372 bp, Artificial Sequence
  Heavy chain
gaggtgcagctggtcgaaagcggcggcggcctggtgcagcccggaggcagcctcaggctgtcttgcgctgcctccggcttcaccttcagcgacgcctggatggattgggtgaggcaggcccccggcaaaggcctggagtgggtggccgagatcaggaacaaggccaacaaccacgccaggcactacgccgagagcgtgaaggggaggttcaccatcagcagggacaacgccaagaacagcctgtacctgcagatgaacagcctgagggccgaggacaccgccgtgtactactgcacccggacctactactacgggagcagctacggctattgcgacgtgtggggccagggcacactagtgaccgtgtccagc

SEQ ID NO: 118 - protein, 124 aa, Artificial Sequence
  Heavy chain
EVQLVESGGGLVQPGGSLRLSCAASGFTFSDAWMDWVRQAPGKGLEWVAEIRNKANNHARHYAESVKGRFTISRDNAKNSLYLQMNSLRAEDTAVYYCTRTYYYGSSYGYCDVWGQGTLVTVSS

SEQ ID NO: 119 - DNA, 372 bp, Artificial Sequence
  Heavy chain
gaggtgcagctggtcgaaagcggcggcggcctggtgcagcccggaggcagcctcaggctgtcttgcgctgcctccggcttcaccagcagcgacgcctggatggattgggtgaggcaggcccccggcaaaggcctggagtgggtggccgagatcaggaacaaggccaacaaccacgccaggcactacgccgagagcgtgaaggggaggttcaccatcagcagggacaacgccaagaacagcctgtacctgcagatgaacagcctgagggccgaggacaccgccgtgtactactgcgcccggacctactactacgggagcagctacggctattgcgacgtgtggggccagggcacactagtgaccgtgtccagc

SEQ ID NO: 120 - protein, 124 aa, Artificial Sequence
  Heavy chain
EVQLVESGGGLVQPGGSLRLSCAASGFTSSDAWMDWVRQAPGKGLEWVAEIRNKANNHARHYAESVKGRFTISRDNAKNSLYLQMNSLRAEDTAVYYCARTYYYGSSYGYCDVWGQGTLVTVSS

SEQ ID NO: 121 - DNA, 372 bp, Artificial Sequence
  Heavy chain
gaggtgcagctggtcgaaagcggcggcggcctggtgcagcccggaggcagcctcaggctgtcttgcgctgcctccggcttcaccagcagcgacgcctggatggattgggtgaggcaggcccccggcaaaggcctggagtgggtggccgagatcaggaacaaggccaacaaccacgccaggcactacgccgagagcgtgaaggggaggttcaccatcagcagggacaacgccaagaacagcctgtacctgcagatgaacagcctgagggccgaggacaccgccgtgtactactgcacccggacctactactacgggagcagctacggctattgcgacgtgtggggccagggcacactagtgaccgtgtccagc

SEQ ID NO: 122 - protein, 124 aa, Artificial Sequence
  Heavy chain
EVQLVESGGGLVQPGGSLRLSCAASGFTSSDAWMDWVRQAPGKGLEWVAEIRNKANNHARHYAESVKGRFTISRDNAKNSLYLQMNSLRAEDTAVYYCTRTYYYGSSYGYCDVWGQGTLVTVSS

SEQ ID NO: 123 - DNA, 372 bp, Artificial Sequence
  Heavy chain
gaggtgcagctggtcgaaagcggcggcggcctggtgcagcccggaggcagcctcaggctgtcttgcgctgcctccggcttcaccagcagcgacgcctggatggattgggtgaggcaggcccccggcaaaggcctggagtgggtggccgagatcaggaacaaggccaacaaccacgccaggcactacgccgagagcgtgaaggggaggttcaccatcagcagggacgacgccaagaacagcctgtacctgcagatgaacagcctgagggccgaggacaccgccgtgtactactgcacccggacctactactacgggagcagctacggctattgcgacgtgtggggccagggcacactagtgaccgtgtccagc

SEQ ID NO: 124 - protein, 124 aa, Artificial Sequence
  Heavy chain
EVQLVESGGGLVQPGGSLRLSCAASGFTSSDAWMDWVRQAPGKGLEWVAEIRNKANNHARHYAESVKGRFTISRDDAKNSLYLQMNSLRAEDTAVYYCTRTYYYGSSYGYCDVWGQGTLVTVSS

SEQ ID NO: 125 - DNA, 372 bp, Artificial Sequence
  Heavy chain
gaggtgcagctggtcgaaagcggcggcggcctggtgcagcccggaggcagcctcaggctgtcttgcgctgcctccggcttcaccagcagcgacgcctggatggattgggtgaggcaggcccccggcaaaggcctggagtgggtggccgagatcaggaacaaggccaacaaccacgccaggcactacgccgagagcgtgaaggggaggttcaccatcagcagggacaacgccaagaacagcgtctacctgcagatgaacagcctgagggccgaggacaccgccgtgtactactgcacccggacctactactacgggagcagctacggctattgcgacgtgtggggccagggcacactagtgaccgtgtccagc

SEQ ID NO: 126 - protein, 124 aa, Artificial Sequence
  Heavy chain
EVQLVESGGGLVQPGGSLRLSCAASGFTSSDAWMDWVRQAPGKGLEWVAEIRNKANNHARHYAESVKGRFTISRDNAKNSVYLQMNSLRAEDTAVYYCTRTYYYGSSYGYCDVWGQGTLVTVSS

SEQ ID NO: 127 - DNA, 372 bp, Artificial Sequence
  Heavy chain
gaggtgcagctggtcgaaagcggcggcggcctggtgcagcccggaggcagcctcaggctgtcttgcgctgcctccggcttcaccagcagcgacgcctggatggattgggtgaggcaggcccccggcaaaggcctggagtgggtggccgagatcaggaacaaggccaacaaccacgccaggcactacgccgagagcgtgaaggggaggttcaccatcagcagggacgacgccaagaacagcgtctacctgcagatgaacagcctgagggccgaggacaccgccgtgtactactgcacccggacctactactacgggagcagctacggctattgcgacgtgtggggccagggcacactagtgaccgtgtccagc

SEQ ID NO: 128 - protein, 124 aa, Artificial Sequence
  Heavy chain
EVQLVESGGGLVQPGGSLRLSCAASGFTSSDAWMDWVRQAPGKGLEWVAEIRNKANNHARHYAESVKGRFTISRDDAKNSVYLQMNSLRAEDTAVYYCTRTYYYGSSYGYCDVWGQGTLVTVSS

SEQ ID NO: 129 - DNA, 327 bp, Artificial Sequence
  Light chain
gacatccagatgacccagagccccagcagcctgagcgctagcgtgggcgacagggtgaccatcacctgcaggacctccgagaacatctacagctacctggcctggtatcagcagaaacccggcaaggcccccaagctcctgatctacaacgccaagacactggccgagggcgtgcccagcaggttctcaggcagcggctctgggaccgacttcaccctgaccatcagcagcctgcagcccgaggacttcgccacctactactgccagcaccactacggaaccccctggaccttcggccagggcaccaagctggagattaagcgtacg

SEQ ID NO: 130 - protein, 109 aa, Artificial Sequence
  Light chain
DIQMTQSPSSLSASVGDRVTITCRTSENIYSYLAWYQQKPGKAPKLLIYNAKTLAEGVPSRFSGSGSGTDFTLTISSLQPEDFATYYCQHHYGTPWTFGQGTKLEIKRT

SEQ ID NO: 131 - DNA, 327 bp, Artificial Sequence
  Light chain
gacatccagatgacccagagccccagcagcctgagcgctagcgtgggcgacagggtgaccatcacctgcaggacctccgagaacatctacagctacctggcctggtatcagcagaaacccggcaaggcccccaagctcctggtgtacaacgccaagacactggccgagggcgtgcccagcaggttctcaggcagcggctctgggaccgacttcaccctgaccatcagcagcctgcagcccgaggacttcgccacctactactgccagcaccactacggaaccccctggaccttcggccagggcaccaagctggagattaagcgtacg

SEQ ID NO: 132 - protein, 109 aa, Artificial Sequence
  Light chain
DIQMTQSPSSLSASVGDRVTITCRTSENIYSYLAWYQQKPGKAPKLLVYNAKTLAEGVPSRFSGSGSGTDFTLTISSLQPEDFATYYCQHHYGTPWTFGQGTKLEIKRT

SEQ ID NO: 133 - DNA, 327 bp, Artificial Sequence
  Light chain
gacatccagatgacccagagccccagcagcctgagcgctagcgtgggcgacagggtgaccatcacctgcaggacctccgagaacatctacagctacctggcctggtatcagcagaaacccggcaaggccccccagctcctggtgtacaacgccaagacactggccgagggcgtgcccagcaggttctcaggcagcggctctgggaccgacttcaccctgaccatcagcagcctgcagcccgaggacttcgccacctactactgccagcaccactacggaaccccctggaccttcggccagggcaccaagctggagattaagcgtacg

SEQ ID NO: 134 - protein, 109 aa, Artificial Sequence
  Light chain
DIQMTQSPSSLSASVGDRVTITCRTSENIYSYLAWYQQKPGKAPQLLVYNAKTLAEGVPSRFSGSGSGTDFTLTISSLQPEDFATYYCQHHYGTPWTFGQGTKLEIKRT

SEQ ID NO: 135 - DNA, 345 bp, Artificial Sequence
  Heavy chain
gaggtgcagctcctggagagcggcgggggcctggtgcagcccggcggcagcctgagactgagctgcgccgcaagcggcttcaccttcagcgacgcctggatggattgggtgaggcaggcccccggcaaaggactggagtgggtcagcgagatcaggcacaaggccaacgaccacgccatcttctacgacgagagcgtgaagggcaggttcaccatctccagggacaacagcaagaacaccctgtacctgcagatgaacagcctgagggccgaagacaccgctgtgtactactgcgccaagccctttgcctactggggccagggaacactagtgaccgtgtccagc

SEQ ID NO: 136 - protein, 115 aa, Artificial Sequence
  Heavy chain
EVQLLESGGGLVQPGGSLRLSCAASGFTFSDAWMDWVRQAPGKGLEWVSEIRHKANDHAIFYDESVKGRFTISRDNSKNTLYLQMNSLRAEDTAVYYCAKPFAYWGQGTLVTVSS

SEQ ID NO: 137 - DNA, 345 bp, Artificial Sequence
  Heavy chain
gaggtgcagctcctggagagcggcgggggcctggtgcagcccggcggcagcctgagactgagctgcgccgcaagcggcttcaccttcagcgacgcctggatggattgggtgaggcaggcccccggcaaaggactggagtgggtcagcgagatcaggcacaaggccaacgaccacgccatcttctacgacgagagcgtgaagggcaggttcaccatctccagggacaacagcaagaacaccctgtacctgcagatgaacagcctgagggccgaagacaccgctgtgtactactgcaccagcccctttgcctactggggccagggaacactagtgaccgtgtccagc

SEQ ID NO: 138 - protein, 115 aa, Artificial Sequence
  Heavy chain
EVQLLESGGGLVQPGGSLRLSCAASGFTFSDAWMDWVRQAPGKGLEWVSEIRHKANDHAIFYDESVKGRFTISRDNSKNTLYLQMNSLRAEDTAVYYCTSPFAYWGQGTLVTVSS

SEQ ID NO: 139 - DNA, 345 bp, Artificial Sequence
  Heavy chain
gaggtgcagctcctggagagcggcgggggcctggtgcagcccggcggcagcctgagactgagctgcgccgcaagcggcttcaccttcagcgacgcctggatggattgggtgaggcaggcccccggcaaaggactggagtgggtcagcgagatcaggcacaaggccaacgaccacgccatcttctacgacgagagcgtgaagggcaggttcaccatctccagggacgacagcaagaacaccctgtacctgcagatgaacagcctgagggccgaagacaccgctgtgtactactgcaccagcccctttgcctactggggccagggaacactagtgaccgtgtccagc

SEQ ID NO: 140 - protein, 115 aa, Artificial Sequence
  Heavy chain
EVQLLESGGGLVQPGGSLRLSCAASGFTFSDAWMDWVRQAPGKGLEWVSEIRHKANDHAIFYDESVKGRFTISRDDSKNTLYLQMNSLRAEDTAVYYCTSPFAYWGQGTLVTVSS

SEQ ID NO: 141 - DNA, 345 bp, Artificial Sequence
  Heavy chain
gaggtgcagctcctggagagcggcgggggcctggtgcagcccggcggcagcctgagactgagctgcgccgcaagcggcttcaccttcagcgacgcctggatggattgggtgaggcaggcccccggcaaaggactggagtgggtcgccgagatcaggcacaaggccaacgaccacgccatcttctacgacgagagcgtgaagggcaggttcaccatctccagggacaacagcaagaacaccgtgtacctgcagatgaacagcctgagggccgaagacaccgctgtgtactactgcaccagcccctttgcctactggggccagggaacactagtgaccgtgtccagc

SEQ ID NO: 142 - protein, 115 aa, Artificial Sequence
  Heavy chain
EVQLLESGGGLVQPGGSLRLSCAASGFTFSDAWMDWVRQAPGKGLEWVAEIRHKANDHAIFYDESVKGRFTISRDNSKNTVYLQMNSLRAEDTAVYYCTSPFAYWGQGTLVTVSS

SEQ ID NO: 143 - DNA, 345 bp, Artificial Sequence
  Heavy chain
gaggtgcagctcctggagagcggcgggggcctggtgcagcccggcggcagcctgagactgagctgcgccgcaagcggcttcaccttcagcgacgcctggatggattgggtgaggcaggcccccggcaaaggactggagtgggtcgccgagatcaggcacaaggccaacgaccacgccatcttctacgacgagagcgtgaagggcaggttcaccatctccagggacgacagcaagaacaccgtgtacctgcagatgaacagcctgagggccgaagacaccgctgtgtactactgcaccagcccctttgcctactggggccagggaacactagtgaccgtgtccagc

SEQ ID NO: 144 - protein, 115 aa, Artificial Sequence
  Heavy chain
EVQLLESGGGLVQPGGSLRLSCAASGFTFSDAWMDWVRQAPGKGLEWVAEIRHKANDHAIFYDESVKGRFTISRDDSKNTVYLQMNSLRAEDTAVYYCTSPFAYWGQGTLVTVSS

SEQ ID NO: 145 - DNA, 327 bp, Artificial Sequence
  Light chain
gacatccagatgacccagagcccctcaagcctgtctgccagcgtgggcgacagggtgaccattacctgcaaggccagccagagcgtcggcaccaccatcgtgtggtaccagcagaaacccggcaaggcccccaagctcctgatctacagcgccagcaacaggcacaccggcgtgcctagcaggtttagcggaagcggcagcggcaccgacttcaccctgacaatcagcagcctgcagcccgaggacttcgccacctactactgccagcagtacaccagctatcccttcaccttcggccagggcaccaagctggagatcaagcgtacg

SEQ ID NO: 146 - protein, 109 aa, Artificial Sequence
  Light chain
DIQMTQSPSSLSASVGDRVTITCKASQSVGTTIVWYQQKPGKAPKLLIYSASNRHTGVPSRFSGSGSGTDFTLTISSLQPEDFATYYCQQYTSYPFTFGQGTKLEIKRT

SEQ ID NO: 147 - DNA, 345 bp, Artificial Sequence; identical to SEQ ID NO: 51
  Heavy chain

SEQ ID NO: 148 - DNA, 327 bp, Artificial Sequence; identical to SEQ ID NO: 53
  Light chain

SEQ ID NO: 149 - DNA, 372 bp, Artificial Sequence; identical to SEQ ID NO: 47
  Heavy chain

SEQ ID NO: 150 - DNA, 327 bp, Artificial Sequence; identical to SEQ ID NO: 49
  Light chain

SEQ ID NO: 151 - DNA, 345 bp, Artificial Sequence
  Heavy chain
gaagtgaagcttgaggagtctggaggaggcttggtgcaacctggaggatccatgaaactctcttgtgctgcctctggaatcacttttagtgatgcctggatggactgggtccgccagtctccagagaaggggcttgagtgggttgctgacattagaaacacagctaataatcatgcaacattctatgctgagtctgtgaaagggaggttcaccatctcaagagatgattccaaaagtactgtctacctgcaaatgaacaccttaagacctgaagacactggcatttattactgtaccagcccttttgcttactggggccaagggactctggtcactgtctctgca

SEQ ID NO: 152 - DNA, 327 bp, Artificial Sequence
  Light chain
gacattgtgatgacccagtctcaaaaattcatgtccacaacagtaggagacagggtcagcatcacctgcaaggccagtcaaaatgtgggtactgctgtagtctggtttcaacagaaaccaggacaatctcctaaactactgatttactcagcatccaatcggtacaccagagtccctgatcgcttcacaggcagtggatctgggacagatttcactctcaccattagcaatgtgcagtctgaagacctggcagattatttctgtcagcaatatgtcaactatccattcacgttcggctcggggacaaaattggaaataaaacgggct

SEQ ID NO: 153 - DNA, 345 bp, Artificial Sequence
  Heavy chain
gaagtgaagtttgaggagtctggaggaggcttggtgcaacctggaggatccatgaaactctcctgtgctgcctccggatttatttttagtgacgcctggatggactgggtccgccagtctccagagaagggacttgagtgggttgctgaaattagacacaaagctaatgattatgcaatattttatgatgagtctgtgaaagggaggttcaccatctcaagagatgattccaaaagtattgtctatctgcaaatgaacaacttaagacctgaagacaccggcatttattattgtaccagtccttttgcttactggggccaagggactctggtcactgtctctgca

SEQ ID NO: 154 - DNA, 327 bp, Artificial Sequence
  Light chain
gacattgtgatgacccagtctcaaaaattcatgtccgtaacagtgggagacagggtcagcatcacctgcaaggccagtcagagtgtgggaactactatagtctggtatcaacagaaaccaggacaatctcctaaattactgatttactcagcatccaatcgacacactggagtccctgatcgcttcacaggcagtggatctgggacagatttcattctcaccattagcaatgtgcagtctgaagacctggcagattatttctgtcagcaatatagcagctatccattcacgttcggctcggggacaaagttggaaataaaacgggct

SEQ ID NO: 155 - DNA, 345 bp, Artificial Sequence
  Heavy chain
gaggtgaagcttgaggagtctggaggaggcttggtgcaacctggaggatccatgaaactctcctgtgctgcctctggattcacttttagtgacgcctggatggactgggtccgccagtctccagagaagggacttgagtgggttgctgaaattagacacaaagctaatgatcatgcaatattttatgatgagtctgtgaaagggaggttcaccatctcaagagatgattccaaaagtattgtctatctgcaaatgaacagcttaagacctgaagacaccggcatttattattgtaccagtccttttgcttactggggccaagggactctggtcactgtctctgca

SEQ ID NO: 156 - DNA, 327 bp, Artificial Sequence
  Light chain
gacattgtgatgacccagtctcaaagattcatgtccgtaacagtgggagacagggtcagcatcacctgcaaggccagtcagagtgtgggtactactatagtctggtatcaacagaaaccaggacaatctcctaaactactgatttactcagcatccactcggtatactggggtccctgatcgcttcacaggcggtggatctgggacagatttcattctcaccattaacaatgtgcagtctgaagacctggcagattatttctgtcagcagtatagcagctatccattcacgttcggctcggggacaaagttggaaataaaacgggct

SEQ ID NO: 157 - DNA, 345 bp, Artificial Sequence
  Heavy chain
gaagtgaagcttgaggagtctggaggaggcttggggcaacctggaggatccatgaaactctcttgtgctgcctctggattcaattttagtgacgcctggatggactgggtccgccagtctccagaaaaggggcttgagtgggttgctgaaattagacacaaagctaatgatcatgcaatattctatgctgagtctgtgaaagggaggttcaccatctcaagagatgattccaaaagtagtgtctacctccaaatgaacagcttaagagctgaagacactggcatttattactgtaccagcccttttgcttactggggccaagggactctggtcactgtctctgca

SEQ ID NO: 158 - protein, 445 aa, Artificial Sequence
  Heavy chain
EVQLLESGGGLVQPGGSLRLSCAASGFTFSDAWMDWVRQAPGKGLEWVAEIRHKANDHAIFYDESVKGRFTISRDDSKNTVYLQMNSLRAEDTAVYYCTSPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - DNA, 1335 bp, Artificial Sequence
  Heavy chain
gaggtgcagctcctggagagcggcgggggcctggtgcagcccggcggcagcctgagactgagctgcgccgcaagcggcttcaccttcagcgacgcctggatggattgggtgaggcaggcccccggcaaaggactggagtgggtcgccgagatcaggcacaaggccaacgaccacgccatcttctacgacgagagcgtgaagggcaggttcaccatctccagggacgacagcaagaacaccgtgtacctgcagatgaacagcctgagggccgaagacaccgctgtgtactactgcaccagcccctttgcctactggggccagggaacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctggccggagcccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

(19) United States
(12) Patent Application Publication
(10) Pub. No.: US 2013/0336989 A1
(43) Pub. Date: Dec. 19, 2013

(54) METHODS OF IDENTIFYING A PATIENT POPULATION

(71) Applicants: Fiona Germaschewski, Stevenage (GB); Jonathan David Larkin, King of Prussia, PA (US); Feng Liu, King of Prussia, PA (US); Thomas Lohr, King of Prussia, PA (US)

(72) Inventors: Fiona Germaschewski, Stevenage (GB); Jonathan David Larkin, King of Prussia, PA (US); Feng Liu, King of Prussia, PA (US); Thomas Lohr, King of Prussia, PA (US)

(73) Assignee: Glaxo Group Limited, Greenford, Middlesex (GB)

(21) Appl. No.: 14/001,202

(22) PCT Filed: Feb. 24, 2012
(86) PCT No.: PCT/US12/26460

§ 371 Date: Aug. 23, 2013

Related U.S. Application Data
(60) Provisional application No. 61/446,259, filed on Feb. 24, 2011.

(51) Int. Cl. G01N 33/573

(52) U.S. Cl. CPC G01N 33/573
USPC 424/158.1; 435/7.4

(57) ABSTRACT

Provided herein is a method for identifying a patient as a candidate for treatment with an aggrecanase inhibitor. Also provided is a method of evaluating the effectiveness of an aggrecanase inhibitor.

### FIELD OF THE INVENTION

This invention relates to methods for identifying patients as candidates for treatment with an aggrecanase inhibitor.

### BACKGROUND OF THE INVENTION

Cartilage is an avascular tissue populated by specialized cells termed chondrocytes, which respond to diverse mechanical and biochemical stimuli. Cartilage is present in the linings of joints, interstitial connective tissues, and basement membranes, and is composed of an extracellular matrix comprised of several matrix components including type II collagen, proteoglycans, fibronectin and laminin.

In normal cartilage, extracellular matrix synthesis is offset by extracellular matrix degradation, resulting in normal matrix turnover. Depending on the signal(s) received, the ensuing response may be either anabolic (leading to matrix production and/or repair) or catabolic (leading to matrix degradation, cellular apoptosis, loss of function, and pain).

In response to injurious compression and/or exposure to inflammatory mediators (e.g. inflammatory cytokines) chondrocytes decrease matrix production and increase production of multiple matrix degrading enzymes. Examples of matrix degrading enzymes include aggrecanases (ADAMTSs) and matrix metalloproteases (MMPs). The activities of these enzymes result in the degradation of the cartilage matrix. Aggrecanases (ADAMTSs), in conjunction with MMPs, degrade aggrecan, an aggregating proteoglycan present in articular cartilage. In osteoarthritic (OA) articular cartilage a loss of proteoglycan staining is observed in the superficial zone in early OA and adjacent to areas of cartilage erosion in moderate to severe OA.

Aggrecan catabolism as mediated by aggrecanase occurs at certain conserved sites in aggrecan. Human ADAMTS4 (shown in FIG. 8 as SEQ ID NO:44) and ADAMTS5 (shown in FIG. 7 as SEQ ID NO:43) have been shown to cleave aggrecan between amino acids E373 and A374 producing the neoepitope ARGSVIL (SEQ ID NO:1). Excessive degradation of extracellular matrix is implicated in the pathogenesis of many diseases and conditions, including pain, chronic pain, neuropathic pain, postoperative pain, rheumatoid arthritis, osteoarthritis, sports injuries, erosive arthritis, ankylosing spondylosis, neuralgia, neuropathies, algesia, nerve injury, ischaemia, neurodegeneration, cartilage degeneration, stroke, incontinence, inflammatory disorders, irritable bowel syndrome, periodontal disease, aberrant angiogenesis, tumor invasion and metastasis, corneal ulceration, complications of diabetes, psoriatic arthritis, inflammatory arthritis and chronic and/or acute kidney disease.

There is a need for identifying those patients who would be the best candidates for treatment with compounds capable of inhibiting aggrecanase activity and cartilage degradation.

### SUMMARY OF THE INVENTION

In one aspect the present invention is directed to a method for identifying a patient as a candidate for treatment with an aggrecanase inhibitor comprising: isolating a biological sample from a patient; and detecting in the sample the presence or absence of at least one aggrecan degradation product; wherein the presence of at least one aggrecan degradation product in the biological sample indicates that the patient is a good candidate for treatment.

In another aspect the present invention is directed to a method of evaluating the effectiveness of an aggrecanase inhibitor comprising obtaining a first measurement of an aggrecan degradation product in a patient; administering an aggrecanase inhibitor to the patient; obtaining a second measurement of the aggrecan degradation product in the patient after administration of the aggrecanase inhibitor; and comparing the first measurement to the second measurement; wherein an inhibition of aggrecanase activity is indicated when the second measurement of the aggrecan degradation product is less than the first measurement of the aggrecan degradation product.

### BRIEF DESCRIPTION OF THE DRAWINGS

FIG. 1: Human OA cartilage explant protocol.

FIG. 2: Individual Human OA Donor Cartilage Explant ARGS Neoepitope Levels and Response to Treatment.

FIG. 3: Data Distribution of ARGS Levels in Human OA Patient Cartilage Explants at Pretreatment Timepoint.

FIG. 4: Effect of 12F4.H4L0 Treatment on ARGS Levels in Human OA Cartilage Explants as a Function of Pretreatment Level Groups.

FIG. 5: Stratification and Effect of Treatment Prediction Based on Human OA Explant Analysis Using ARGS Neoepitope Levels.

FIG. 6: ARGS Neoepitope Levels in OA Patient and Healthy Volunteer Serum - Pilot Study Ranges.

FIG. 7: ARGS Neoepitope Levels in OA Patient and Healthy Volunteer Plasma - Pilot Study Ranges.

FIG. 8: ARGS Neoepitope Levels in OA Patient and Healthy Volunteer Urine - Pilot Study Ranges.

FIG. 9: ARGS Neoepitope Levels in OA Patient Samples Compared to RA Patient Serum and Healthy Volunteer Serum.

FIG. 10: ARGS Neoepitope Levels in OA Surgical Patient Samples Compared to Non Surgical OA and RA patient synovial fluid

FIG. 11: ARGS Neoepitope Levels in OA Versus RA Patient Urine Compared to Healthy Volunteers.

FIG. 12: Amino Acid sequence of human ADAMTS5 (SEQ ID NO:43).

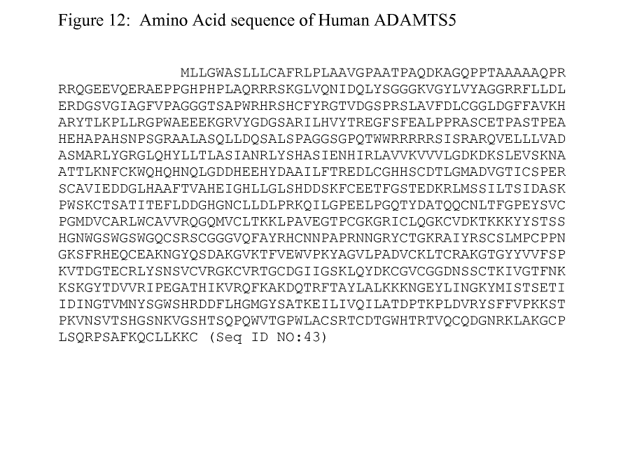

FIG. 13: Amino Acid sequence of Human ADAMTS4 (SEQ ID NO:44).

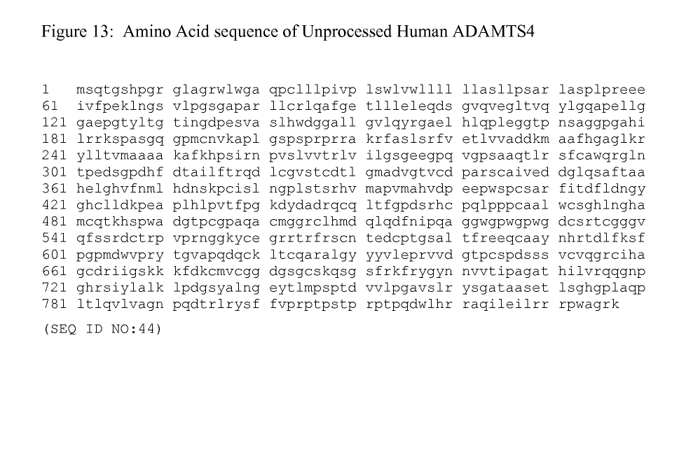

FIG. 14: Aggrecanase Cleavage Sites

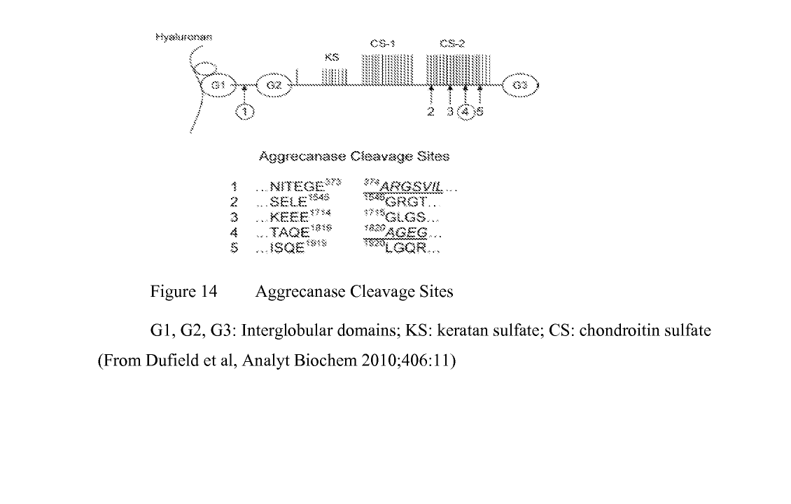

FIG. 15: Aggrecan Neoepitope Assay Format

FIG. 16: Correlation of mean ARGS levels in matched healthy donor (n=20) and OA (n=5) serum and plasma

### DETAILED DESCRIPTION OF THE INVENTION

In one aspect the present invention is directed to a method for identifying a patient as a candidate for treatment with an aggrecanase inhibitor comprising: isolating a biological sample from a patient; and detecting in the sample the presence or absence of at least one aggrecan degradation product; wherein the presence of at least one aggrecan degradation product in the biological sample indicates that the patient is a good candidate for treatment.

In another aspect the present invention is directed to a method of evaluating the effectiveness of an aggrecanase inhibitor comprising obtaining a first measurement of an aggrecan degradation product in a patient; administering an aggrecanase inhibitor to the patient; obtaining a second measurement of the aggrecan degradation product in the patient after administration of the aggrecanase inhibitor; and comparing the first measurement to the second measurement; wherein an inhibition of aggrecanase activity is indicated when the second measurement of the aggrecan degradation product is less than the first measurement of the aggrecan degradation product.

In one embodiment, the aggrecanase inhibitor inhibits the activity of an aggrecanse selected from the group consisting of ADAMTS1, ADAMTS4, ADAMTS5, ADAMTS9, and ADAMTS15.

In one embodiment, the aggrecanase inhibitor is GSK571949 (CAS number 329040-94-0) below.

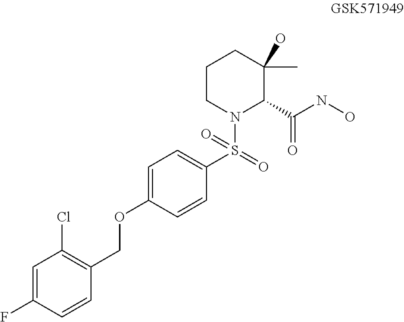 [SMILES: C[C@@]1(O)CCCN(S(=O)(=O)c2ccc(OCc3ccc(F)cc3Cl)cc2)[C@H]1C(=O)NO]

In one embodiment, the aggrecanase inhibitor is an antigen binding protein. In one embodiment the antigen binding protein is an antibody or a fragment thereof.

In another embodiment, the antigen binding protein comprises at least one complementarity determining region. In some instances, the antigen binding protein is a monoclonal antibody comprising a heavy chain comprising CDRH1, CDRH2 and CDRH3 and a light chain comprising CDRL1, CDRL2 and CDRL3, wherein the complementarity determining regions (CDRs) of the heavy chain are selected from the group of:
CDRH1 having at least about 80% sequence identity to amino acid sequence DAWMD (SEQ ID NO:2);
CDRH2 having at least about 70, 75, 80, 85, 90, 95, or 98% sequence identity to amino acid sequence EIRHKANDHAIFYXESVKG (SEQ ID NO:3); and
CDRH3 having at least about 70, 75, 80, 85, 90, 95, or 98% sequence identity to amino acid sequence TYYYGSSYGYCDV (SEQ ID NO:4) or PFAY (SEQ ID NO:5); and
   the complementarity determining regions of the light chain are selected from the group of:
CDRL1 having at least about 70, 75, 80, 85, 90, 95, or 98% sequence identity to amino acid sequence KASQSVGTTIV (SEQ ID NO:6) or RTSENIYSYLA (SEQ ID NO:7);
CDRL2 having at least about 70, 75, 80, 85, 90, 95, or 98% sequence identity to amino acid sequence NAKTLAE (SEQ ID NO:8) or SASNRXT (SEQ ID NO:9); and
CDRL3 having at least about 70, 75, 80, 85, 90, 95, or 98% sequence identity to amino acid sequence QQYSSYPFT(SEQ ID NO:10) or QHHYGTPWT ((SEQ ID NO:11).

In one embodiment, CDRH2 has at least about 70, 75, 80, 85, 90, 95, or 98% sequence identity an amino acid sequence selected from EIRHKANDHAIFYAESVKG (SEQ ID NO:12), EIRNKANNHARHYAESVKG (SEQ ID NO:13), EIRHKANDYAIFYDESVKG (SEQ ID NO:14), EIRHKANDHAIFYDESVKG (SEQ ID NO:15), DIRNTANNHATFYAESVKG (SEQ ID NO:16), and EIRHKANDHAIFYDESVKG (SEQ ID NO:17). In one embodiment, CDRH3 comprises the amino acid sequence, PFAY (SEQ ID NO:5).

In yet another embodiment, the antigen binding proteins are monoclonal antibodies comprising a heavy chain comprising CDRH1, CDRH2 and CDRH3 and a light chain comprising CDRL1, CDRL2 and CDRL3, wherein the complementarity determining regions (CDRs) of the heavy chain are selected from:
CDRH1 is amino acid sequence DAWMD (SEQ ID NO:2);
CDRH2 is select from amino acid sequence EIRHKANDHAIFYAESVKG (SEQ ID NO:12), EIRNKANNHARHYAESVKG (SEQ ID NO:13), EIRHKANDYAIFYDESVKG (SEQ ID NO:14), EIRHKANDHAIFYDESVKG (SEQ ID NO:15), DIRNTANNHATFYAESVKG (SEQ ID NO:16), or EIRHKANDHAIFYDESVKG (SEQ ID NO:17); and
CDRH3 is TYYYGSSYGYCDV (SEQ ID NO:18) or PFAY (SEQ ID NO:5); and
   the complementarity determining regions of the light chain are selected from:
CDRL1 is select from amino acid sequence KASQSVGTTIV (SEQ ID NO:19), RTSENIYSYLA (SEQ ID NO:20), or KASQNVGTAVV (SEQ ID NO:21);
CDRL2 is select from amino acid sequence NAKTLAE (SEQ ID NO:22), SASNRHT (SEQ ID NO:23), SASTRYT (SEQ ID NO:24), or SASNRYT (SEQ ID NO:25); and
CDRL3 is select from amino acid sequence QQYSSYPFT (SEQ ID NO:26), QHHYGTPWT (SEQ ID NO:27), QQYVNYPFT (SEQ ID NO:28), or QQYTSYPFT (SEQ ID NO:29).

Thus, in one embodiment, the antigen binding protein comprises an isolated monoclonal antibody is provided comprising six CDRs wherein CDRH1 is DAWMD (SEQ ID NO:2), CDRH2 is EIRNKANNHARHYAESVKG (SEQ ID NO:13), and CDRH3 is TYYYGSSYGYCDV (SEQ ID NO:18) and CDRL1 is RTSENIYSYLA (SEQ ID NO:20), CDRL2 is NAKTLAE (SEQ ID NO:22) and CDRL3 is QHHYGTPWT (SEQ ID NO:27). In another embodiment, the antigen binding protein comprises an isolated monoclonal antibody is provided comprising six CDRs wherein CDRH1 is DAWMD (SEQ ID NO:2), CDRH2 is EIRHKANDHAIFYDESVKG (SEQ ID NO:15), and CDRH3 is PFAY (SEQ ID NO:5) and CDRL1 is KASQSVGTTIV (SEQ ID NO:19), CDRL2 is SASNRHT (SEQ ID NO:23) and CDRL3 is QQYTSYPFT (SEQ ID NO:29).

In yet another embodiment, the antigen binding proteins are monoclonal antibodies comprising a heavy chain comprising CDRH1, CDRH2 and CDRH3 and a light chain comprising CDRL1, CDRL2 and CDRL3, wherein the complementarity determining regions (CDRs) of the heavy chain are selected from:
CDRH1 is amino acid sequence DAWMD (SEQ ID NO:2), wherein any amino acid of SEQ ID NO: 2 is substituted at one position by an amino acid selected from histidine, isoleucine, leucine, lysine, methionine, phenylalanine, threonine, tryptophan, valine, alanine, arginine, aspartic acid, cysteine, cystine, glutamic acid, glutamine, glycine, ornithine, proline, serine, taurine, and tyrosine;
CDRH2 is select from amino acid sequence EIRHKANDHAIFYAESVKG (SEQ ID NO:12), EIRNKANNHARHYAESVKG (SEQ ID NO:13),

EIRHKANDYAIFYDESVKG (SEQ ID NO:14), EIRHKANDHAIFYDESVKG (SEQ ID NO:15), DIRNTANNHATFYAESVKG (SEQ ID NO:16), or EIRHKANDHAIFYDESVKG (SEQ ID NO:17) , wherein any amino acid of SEQ ID NOS: 12-17 is substituted at one position by an amino acid selected from histidine, isoleucine, leucine, lysine, methionine, phenylalanine, threonine, tryptophan, valine, alanine, arginine, aspartic acid, cysteine, cystine, glutamic acid, glutamine, glycine, ornithine, proline, serine, taurine, and tyrosine; and
CDRH3 is TYYYGSSYGYCDV (SEQ ID NO:18) or PFAY (SEQ ID NO:5) , wherein any amino acid of SEQ ID NOS: 18 and 5 is substituted at one position by an amino acid selected from histidine, isoleucine, leucine, lysine, methionine, phenylalanine, threonine, tryptophan, valine, alanine, arginine, aspartic acid, cysteine, cystine, glutamic acid, glutamine, glycine, ornithine, proline, serine, taurine, and tyrosine; and
   the complementarity determining regions of the light chain are selected from:
CDRL1 is select from amino acid sequence KASQSVGTTIV (SEQ ID NO:19), RTSENIYSYLA (SEQ ID NO:20), or KASQNVGTAVV (SEQ ID NO:21) , wherein any amino acid of SEQ ID NO: 19-21 is substituted at one position by an amino acid selected from histidine, isoleucine, leucine, lysine, methionine, phenylalanine, threonine, tryptophan, valine, alanine, arginine, aspartic acid, cysteine, cystine, glutamic acid, glutamine, glycine, ornithine, proline, serine, taurine, and tyrosine;
CDRL2 is select from amino acid sequence NAKTLAE (SEQ ID NO:22), SASNRHT (SEQ ID NO:23), SASTRYT (SEQ ID NO:24), or SASNRYT (SEQ ID NO:25) , wherein any amino acid of SEQ ID NO: 22-25 is substituted at one position by an amino acid selected from histidine, isoleucine, leucine, lysine, methionine, phenylalanine, threonine, tryptophan, valine, alanine, arginine, aspartic acid, cysteine, cystine, glutamic acid, glutamine, glycine, ornithine, proline, serine, taurine, and tyrosine; and
CDRL3 is select from amino acid sequence QQYSSYPFT (SEQ ID NO:26), QHHYGTPWT (SEQ ID NO:27), QQYVNYPFT (SEQ ID NO:28), or QQYTSYPFT (SEQ ID NO:29), wherein any amino acid of SEQ ID NO: 26-29 is substituted at one position by an amino acid selected from histidine, isoleucine, leucine, lysine, methionine, phenylalanine, threonine, tryptophan, valine, alanine, arginine, aspartic acid, cysteine, cystine, glutamic acid, glutamine, glycine, ornithine, proline, serine, taurine, and tyrosine.

In certain embodiments, Thr4 of NAKTLAE (SEQ ID NO:22) is leucine, isoleucine or methionine. In certain embodiments, His3 of QHHYGTPWT (SEQ ID NO:27) is valine. In certain embodiments, Gly5 of QHHYGTPWT (SEQ ID NO:27) is tryptophan, tyrosine, phenylalanine, or methionine. In certain embodiments, His9 of EIRNKANNHARHYAESVKG (SEQ ID NO:13) is phenylalanine or tyrosine. In certain embodiments, Ser6 of TYYYGSSYGYCDV (SEQ ID NO:18) is phenylalanine or tyrosine.

The CDRs L1, L2, L3, H1 and H2 tend to structurally exhibit one of a finite number of main chain conformations. The particular canonical structure class of a CDR is defined by both the length of the CDR and by the loop packing, determined by residues located at key positions in both the CDRs and the framework regions (structurally determining residues or SDRs). Martin and Thornton (1996; J Mol Biol 263:800-815) have generated an automatic method to define the “key residue” canonical templates. Cluster analysis is used to define the canonical classes for sets of CDRs, and canonical templates are then identified by analysing buried hydrophobics, hydrogen-bonding residues, and conserved glycines and prolines. The CDRs of antibody sequences can be assigned to canonical classes by comparing the sequences to the key residue templates and scoring each template using identity or similarity matrices.

Examples of CDR canonicals are given below. The amino acid numbering used is Kabat.

Examples of canonicals for CDRH1 as set out in SEQ ID NO:144, or a variant thereof are: Ala 32 is substituted for Ile, His, Tyr, Phe, Thr, Asn, Cys, Glu or Asp; Trp 33 is substituted for Tyr, Ala, Gly, Thr, Leu or Val; Met 34 is substituted for Ile, Val or Trp; and Asp 35 is substituted for His, Glu, Asn, Gln, Ser, Tyr or Thr.

Examples of canonicals for CDRH2 as set out in SEQ ID NO:144, or a variant thereof are: Glu 50 is substituted for Arg or Gln; and Ile 51 is substituted for Leu, Val, Thr, Ser or Asn.

Examples of canonicals for CDRH3 as set out in SEQ ID NO:144, or a variant thereof are: Tyr 102 is substituted for His, Val, Ile, Ser, Asp or Gly.

Examples of canonicals for CDRL1 as set out in SEQ ID NO:146, or a variant thereof are: Ser 28 is substituted for Asn, Asp, Thr or Glu; Val 29 is substituted for Ile; Gly 30 is substituted for Asp, Leu, Tyr, Val, Ile, Ser, Asn, Phe, His or Thr; Thr 31 is substituted for Ser, Asn, Lys or Gly; Thr 32 is substituted for Phe, Tyr, Asn, Ala, His, Ser or Arg; Ile 33 is substituted for Met, Leu, Val or Phe; and Val 34 is substituted for Ala, Gly, Asn, Ser, His or Phe.

Examples of canonicals for CDRL3 as set out in SEQ ID NO:146, or a variant thereof are: Gln 89 is substituted for Ser, Gly, Phe or Leu; Gln 90 is substituted for Asn or His; Tyr 91 is substituted for Asn, Phe, Gly, Ser, Arg, Asp, His, Thr or Val; Thr 92 is substituted for Asn, Tyr, Trp, Ser, Arg, Gln, His, Ala or Asp; Ser 93 is substituted for Gly, Asn, Thr, Arg, Glu, Ala or His; Tyr 94 is substituted for Asp, Thr, Val, Leu, His, Asn, Ile, Tip, Pro or Ser; and Phe 96 is substituted for Pro, Leu, Tyr, Arg, Ile or Trp.

In other aspects the antigen binding protein is a Fab or F(ab)₂ fragment. In another embodiment, the first immunoglobulin variable domain is a single chain variable domain.

In one embodiment the antigen binding protein comprises an antibody as described herein and comprising a constant domain region such that the antibody has reduced ADCC and/or complement activation or effector functionality. In one such embodiment the constant domain may comprise a naturally disabled constant region of IgG2 or IgG4 isotype or a mutated IgG1 constant domain. Examples of suitable modifications are described in EP0307434. One example comprises the substitutions of alanine residues at positions 235 and 237 (EU index numbering). In one embodiment, such an antibody comprises the heavy chain of SEQ ID NO:158.

In one embodiment the antigen binding protein or a fragment thereof comprises an antibody V_{H} domain comprising an amino acid sequence selected from the group consisting of SEQ ID NO: 76, 80, 116, 118, 120, 122, 124, 126, 128, 136, 138, 140, 142, and 144.

In one embodiment the antigen binding protein or a fragment thereof comprises an antibody V_{L} domain comprising an amino acid sequence selected from the group consisting of SEQ ID NO: 78, 82, 130, 132, 134, and 146.

In one embodiment the antigen binding protein or a fragment thereof comprises an antibody V_{H} domain comprising an amino acid sequence selected from the group consisting of SEQ ID NO: 76, 80, 116, 118, 120, 122, 124, 126, 128, 136, 138, 140, 142, and 144 and a V_{L} domain comprising an amino acid sequence selected from the group consisting of SEQ ID NO: 78, 82, 130, 132, 134, and 146.

In one embodiment the antigen binding protein or a fragment thereof comprises an antibody V_{H} domain comprising SEQ ID NO: 76 and a V_{L} domain comprising SEQ ID NO: 78.

In one embodiment the antigen binding protein or a fragment thereof comprises an antibody V_{H} domain comprising SEQ ID NO: 80 and a V_{L} domain comprising SEQ ID NO: 82.

In one embodiment the antigen binding protein or a fragment thereof comprises an antibody V_{H} domain comprising an amino acid sequence selected from the group consisting of SEQ ID NO: 116, 118, 120, 122, 124, 126, and 128 and a V_{L} domain comprising an amino acid sequence selected from the group consisting of SEQ ID NO: 130, 132, and 134.

In one embodiment the antigen binding protein or a fragment thereof comprises an antibody V_{H} domain comprising an amino acid sequence selected from the group consisting of SEQ ID NO: 136, 138, 140, 142, and 144 and a V_{L} domain comprising SEQ ID NO: 146.

In one embodiment the antigen binding protein or a fragment thereof comprises an antibody heavy chain comprising an amino acid sequence selected from the group consisting of SEQ ID NO: 68, 72, 84, 86, 88, 90, 92, 94, 96, 104, 106, 108, 110, 112, and 158.

In one embodiment the antigen binding protein or a fragment thereof comprises an antibody light chain comprising an amino acid sequence selected from the group consisting of SEQ ID NO: 70, 74, 98, 100, 102, and 114.

In one embodiment the antigen binding protein or a fragment thereof comprises an antibody heavy chain comprising an amino acid sequence selected from the group consisting of SEQ ID NO: 68, 72, 84, 86, 88, 90, 92, 94, 96, 104, 106, 108, 110, 112, and 158 and an antibody light chain comprising an amino acid sequence selected from the group consisting of SEQ ID NO: 70, 74, 98, 100, 102, and 114.

In one embodiment the antigen binding protein or a fragment thereof comprises an antibody heavy chain comprising SEQ ID NO: 68 and an antibody light chain comprising SEQ ID NO: 70.

In one embodiment the antigen binding protein or a fragment thereof comprises an antibody heavy chain comprising SEQ ID NO: 72 and an antibody light chain comprising SEQ ID NO: 74.

In one embodiment the antigen binding protein or a fragment thereof comprises an antibody heavy chain comprising an amino acid sequence selected from the group consisting of SEQ ID NO: 84, 86, 88, 90, 92, 94, and 96 and an antibody light chain comprising an amino acid sequence selected from the group consisting of SEQ ID NO: 98, 100, and 102.

In one embodiment the antigen binding protein or a fragment thereof comprises an antibody heavy chain comprising an amino acid sequence selected from the group consisting of SEQ ID NO: 104, 106, 108, 110, 112, and 158 and an antibody light chain comprising SEQ ID NO: 114.

In one embodiment the antigen binding protein or a fragment thereof comprises an antibody that competes for binding to ADAMTS5 with any one of the antibodies listed in Table 1. These include the antibodies 1G10.1C9, 2D3.1D4, 3A12.1D7, 5F10.1H6, 11F12.1D12, 12F4.1H7, and 7B4.1E11.

In one embodiment, the at least one aggrecan degradation product comprises the neoepitope ARGSVIL.

In one embodiment, the patient is suffering from a disease or condition selected from the group consisting of chronic pain, neuropathic pain, postoperative pain, rheumatoid arthritis, osteoarthritis, neuralgia, neuropathies, algesia, nerve injury, ischaemia, neurodegeneration, cartilage degeneration, stroke, incontinence, inflammatory disorders, irritable bowel syndrome, periodontal disease, aberrant angiogenesis, tumor invasion and metastasis, corneal ulceration, complications of diabetes, psoriatic arthritis, inflammatory arthritis and chronic and/or acute kidney disease.

In one embodiment, the aggrecan degradation product is detected using an antibody or a fragment thereof. In another embodiment the aggrecan degradation product is detected using mass spectrometry.

In one embodiment, the antibody or a fragment thereof used to detect the aggrecan degradation product is OA-1. In one embodiment, the biological sample is human blood, plasma, serum, saliva, synovial fluid, interstitial fluid, urine or heart tissue serum.

In one embodiment, the biological sample is human serum and the aggrecan degradation product comprising the neoepitope ARGSVIL is present at a concentration of at least about 0.5, 1, 2, 3, 4, 5, 6, 7, 8, 9, 10, 11, 12, 13, 14, 15, 16, 17, 18, 19, 20 ng/ml. In one embodiment the neoepitope ARGSVIL is present at a concentration of at least about 6 ng/ml.

In one embodiment, the biological sample is human plasma and the aggrecan degradation product comprising the neoepitope ARGSVIL is present at a concentration of at least about 0.5, 1, 2, 3, 4, 5, 6, 7, 8, 9, 10, 11, 12, 13, 14, 15, 16, 17, 18, 19, 20 ng/ml. In one embodiment, the neoepitope ARGSVIL is present at a concentration of at least about 10 ng/ml.

In one embodiment, the biological sample is urine and the aggrecan degradation product comprising the neoepitope ARGSVIL is present at a concentration of at least about 0.5, 1, 2, 3, 4, 5, 6, 7, 8, 9, 10, 11, 12, 13, 14, 15, 16, 17, 18, 19, 20 ng/ml. In one embodiment, the neoepitope ARGSVIL is present at a concentration of at least about 5 ng/ml.

“Polynucleotide” generally refers to any polyribonucleotide or polydeoxribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as posttranslational processing, or by chemical modification techniques that are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. See, for instance, PROTEINS—STRUCTURE AND MOLECULAR PROPERTIES, 2nd Ed., T. E. Creighton, W. H. Freeman and Company, New York, 1993 and Wold, F., Posttranslational Protein Modifications: Perspectives and Prospects, pgs. 1-12 in POSTTRANSLATIONAL COVALENT MODIFICATION OF PROTEINS, B. C. Johnson, Ed., Academic Press, New York, 1983; Seifter, et al., “Analysis for protein modifications and nonprotein cofactors”, Meth. Enzymol. (1990) 182:626-646 and Rattan, et al., “Protein Synthesis: Posttranslational Modifications and Aging”, Ann NY Acad Sci (1992) 663:48-62.

“Variant” as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated,” including, but not limited to, when such polynucleotide or polypeptide is introduced back into a cell.

An “isolated” or “substantially pure” nucleic acid or polynucleotide (e.g., an RNA, DNA or a mixed polymer) is one which is substantially separated from other cellular components that naturally accompany the native polynucleotide in its natural host cell, e.g., ribosomes, polymerases and genomic sequences with which it is naturally associated. The term embraces a nucleic acid or polynucleotide that (1) has been removed from its naturally occurring environment, (2) is not associated with all or a portion of a polynucleotide in which the “isolated polynucleotide” is found in nature, (3) is operatively linked to a polynucleotide which it is not linked to in nature, or (4) does not occur in nature. The term “isolated” or “substantially pure” also can be used in reference to recombinant or cloned DNA isolates, chemically synthesized polynucleotide analogs, or polynucleotide analogs that are biologically synthesized by heterologous systems.

However, “isolated” does not necessarily require that the nucleic acid or polynucleotide so described has itself been physically removed from its native environment. For instance, an endogenous nucleic acid sequence in the genome of an organism is deemed “isolated” herein if a heterologous sequence is placed adjacent to the endogenous nucleic acid sequence, such that the expression of this endogenous nucleic acid sequence is altered, for example, increased, decreased or eliminated. In this context, a heterologous sequence is a sequence that is not naturally adjacent to the endogenous nucleic acid sequence, whether or not the heterologous sequence is itself endogenous (originating from the same host cell or progeny thereof) or exogenous (originating from a different host cell or progeny thereof). By way of example, a promoter sequence can be substituted (e.g., by homologous recombination) for the native promoter of a gene in the genome of a host cell, such that this gene has an altered expression pattern. This gene would now become “isolated” because it is separated from at least some of the sequences that naturally flank it.

A nucleic acid is also considered “isolated” if it contains any modifications that do not naturally occur to the corresponding nucleic acid in a genome. For instance, an endogenous coding sequence is considered “isolated” if it contains an insertion, deletion or a point mutation introduced artificially, e.g., by human intervention. An “isolated nucleic acid” also includes a nucleic acid integrated into a host cell chromosome at a heterologous site and a nucleic acid construct present as an episome. Moreover, an “isolated nucleic acid” can be substantially free of other cellular material, or substantially free of culture medium when produced by recombinant techniques, or substantially free of chemical precursors or other chemicals when chemically synthesized.

As used herein “inflammatory mediators” include any compound capable of triggering an inflammatory process. The term inflammation generally refers to the process of reaction of vascularized living tissue to injury. This process includes but is not limited to increased blood flow, increased vascular permeability, and leukocytic exudation. Because leukocytes recruited into inflammatory reactions can release potent enzymes and oxygen free radicals (i.e. inflammatory mediators), the inflammatory response is capable of mediating considerable tissue damage. Examples of inflammatory mediators include, but are not limited to prostaglandins (e.g. PGE2), leukotrienes (e.g. LTB4), inflammatory cytokines, such as tumour necrosis factor alpha (TNFα), interleukin 1 (IL-1), and interleukin 6 (IL-6); nitric oxide (NO), metalloproteinases, and heat shock proteins.

As used herein “matrix protein” includes proteins released from cells to form the extracellular matrix of cartilage. The extracellular matrix of cartilage consists of proteoglycans, belonging to several distinct proteoglycan families. These include, but are not limited to, perlecan and the hyalectans, exemplified by aggrecan and versican, and the small leucine-rich family of proteoglycans, including decorin, biglycan and fibromodulin. The extracellular matrix also consists of hybrid collagen fibers comprised of three collagen isotypes, namely type II, type IX, and type XI collagens, along with accessory proteins such as cartilage oligeromeric matrix protein (COMP), link protein, and fibronectin. Cartilage also contains hyaluronin which forms a noncovalent association with the hyalectins. In addition, a specialized pericellular matrix surrounds the chondrocyte which consists of proteoglycans, type VI collagen and collagen receptor proteins, such as anchorin.

As used herein “matrix degrading enzymes” refers to enzymes able to cleave extracellular matrix proteins. Cartilage extracellular matrix turnover is regulated by matrix metalloproteases (MMPs) which are synthesized as latent proenzymes that require activation in order to degrade cartilage extracellular matrix proteins. Three classes of enzymes are believed to regulate the turnover of extracellular matrix proteins, namely collagenases (including, but not limited to, MMP-13), responsible for the degradation of native collagen fibers, stromelysins (including, but not limited to, MMP-3) which degrade proteoglycan and type IX collagen, and gelatinases (including, but not limited to, MMP-2 and MMP-9) which degrade denatured collagen. The matrix degrading enzyme group that appears most relevant in cartilage degradation in OA includes a subgroup of metalloproteinases called ADAMTS, because they possess disintegrin and metalloproteinase domains and a thrombospondin motif in their structure. ADAMTS4 (aggrecanase-1) has been reported to be elevated in OA joints and along with ADAMTS-5 (aggrecanase-2) have been shown to be expressed in human osteoarthritic cartilage. These enzymes appear to be responsible for aggrecan degradation without MMP participation.

As used herein, “reduce” or “reducing” aggrecanase activity refers to a decrease in any and/or all of the activities associated with at least one naturally occurring aggrecanase, including but not limited to ADAMTS4 and ADAMTS5. For example “reducing” at least one ADAMTS5 activity refers to a decrease in any and/or all of the activities associated with naturally occurring ADAMTS5. By way of example, reducing ADAMTS5 in a mammal activity can be measured after administration of at least one polypeptide capable of binding to ADAMTS5 to a subject and compared with ADAMTS5 activity in the same subject prior to the administration of the polypeptide capable of binding to ADAMTS5 or in comparison to a second subject who is administered placebo. As used herein, “reducing” at least one ADAMTS5 includes the reduction of at least one or more enzyme activity. A reduction in at least one ADAMTS5 activity includes a complete abrogation of at least one ADAMTS5. Also included within this definition is a reduced amount of at least one enzyme activity. That is, ADAMTS5 may have more than one activity which is maintained the while a second activity of the same enzyme is reduced.

As used herein “diseases associated with cartilage degradation” include, but are not limited to cancer, pain, chronic pain, neuropathic pain, postoperative pain, osteoarthritis, sports injuries, erosive arthritis, rheumatoid arthritis, psoriatic arthritis, Lyme arthritis, juvenile arthritis, ankylosing spondylosis, neuralgia, neuropathies, algesia, nerve injury, ischaemia, neurodegeneration, inflammatory diseases, cartilage degeneration, diseases affecting the larynx, trachea, auditory canal, intervertebral discs, ligaments, tendons, joint capsules or bone development, invertebral disc degeneration, osteopenia, or periodontal diseases, acute joint injury, and/or a disease related to joint destruction.

As used herein “co-administration” or “co-administering” as used herein refers to administration of two or more compounds to the same patient. Co-administration of such compounds may be simultaneous or at about the same time (e.g., within the same hour) or it may be within several hours or days of one another. For example, a first compound may be administered once weekly while a second compound is co-administered daily.

As used herein “attenuate” or “attenuating” refers to a normalization (i.e., either an increase or decrease) of the amount of matrix degrading enzyme, inflammatory mediator, or matrix protein produced and/or released by a cell, following exposure to a catabolic stimulus. For example, following exposure to IL-1 chondrocyte production of matrix proteins, such as proteoglycans, are reduced, while production of matrix degrading enzymes (e.g. MMP-13, ADAMTS4) and reactive oxygen species (e.g. NO) are increased. Attenuation refers to the normalization of these diverse responses to levels observed in the absence of a catabolic stimulus.

As used herein, the term “antibody” includes immunoglobulin molecules comprising four polypeptide chains, two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chains of antibodies (immunoglobulins) from any vertebrate species can be assigned to one of two clearly distinct types, called kappa (κ) and lambda (λ), based on the amino acid sequences of their constant domains. The variable regions of kappa light chains are referred to herein as VK. The expression V_{L}, as used herein, is intended to include both the variable regions from kappa-type light chains (VK) and from lambda-type light chains. The light chain constant region is comprised of one domain, CL. The V_{H} and V_{L} regions include regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1-CDR1-FR2-CDR2-FR3-CDR3-FR4.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known. The present invention includes antibodies of any of the aforementioned classes or subclasses (isotypes).

The term “antibody” as used herein is also intended to encompass antibodies, digestion fragments, specified portions and variants thereof, including antibody mimetics or comprising portions of antibodies that mimic the structure and/or function of an antibody or specified fragment or portion thereof, including single chain antibodies and fragments thereof; each containing at least one CDR. Functional fragments include antigen binding fragments that bind to an ADAMTS5 antigen. For example, antibody fragments capable of binding to ADAMTS5or a portion thereof, including, but not limited to Fab (e.g., by papain digestion), facb (e.g., by plasmin digestion), pFc′ (e.g., by pepsin or plasmin digestion), Fd (e.g., by pepsin digestion, partial reduction and reaggregation), FIT or scFv (e.g., by molecular biology techniques) fragments, are encompassed by the present invention. Antibody fragments are also intended to include, e.g., domain deleted antibodies, diabodies, linear antibodies, single-chain antibody molecules, and multispecific antibodies formed from antibody fragments.

The term “monoclonal antibody,” as used herein, refers to an antibody obtained from a population of substantially homogeneous antibodies, e.g., the individual antibodies comprising the population are substantially identical except for possible naturally occurring mutations or minor post-translational variations that may be present. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. The monoclonal antibodies of the present invention are preferably made by recombinant DNA methods or are obtained by screening methods as described elsewhere herein.

The term “monoclonal antibodies,” as used herein, includes “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species (e.g., mouse or rat) or belonging to another antibody class or subclass, as well as fragments of such antibodies, so long as they exhibit the desired biological activity (Morrison et al., Proc. Natl. Acad. Sci. USA 81:6851-6855 (1984)). Chimeric antibodies of interest herein include “primatized” antibodies comprising variable domain antigen-binding sequences derived from a non-human primate (e.g., Old World Monkey, such as baboon, rhesus or cynomolgus monkey) and human constant region sequences (U.S. Pat. No. 5,693,780).

Thus, the present invention includes, for example, chimeric monoclonal antibodies comprising a chimeric heavy chain and/or a chimeric light chain. The chimeric heavy chain may comprise any of the heavy chain variable (V_{H}) regions described herein or mutants or variants thereof fused to a heavy chain constant region of a non-human or a human antibody. The chimeric light chain may comprise any of the light chain variable (V_{L}) regions described herein or mutants or variants thereof fused to a light chain constant region of a non-human or a human antibody.

The term “human antibody,” as used herein, includes antibodies having variable and constant regions corresponding to human germline immunoglobulin sequences as described by Kabat et al. (See Kabat, et al. (1991) Sequences of Proteins of Immunological Interest, Fifth Edition, U.S. Department of Health and Human Services, NIH Publication No. 91-3242). The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. The human antibody can have at least one position replaced with an amino acid residue, e.g., an activity enhancing amino acid residue which is not encoded by the human germline immunoglobulin sequence. In the context of the present invention, the human antibody can have up to twenty positions replaced with amino acid residues which are not part of the human germline immunoglobulin sequence. In other embodiments, up to ten, up to five, up to three or up to two positions are replaced. However, the term “human antibody,” as used herein, is not intended to include antibodies in which CDR sequences derived from the germline of another mammalian species, such as a mouse, have been grafted onto human framework sequences.

The phrase “recombinant human antibody” includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal that is transgenic for human immunoglobulin genes, or antibodies prepared, expressed, created or isolated by any other means that involves splicing of human immunoglobulin gene sequences to other DNA sequences. Such recombinant human antibodies have variable and constant regions derived from human germline immunoglobulin sequences (See Kabat, E. A., et al. (1991) Sequences of Proteins of Immunological Interest, Fifth Edition, U.S. Department of Health and Human Services, NIH Publication No. 91-3242). According to the present invention, recombinant human antibodies include human germline immunoglobulin sequence that have been subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo. In certain embodiments, however, such recombinant antibodies are the result of selective mutagenesis approach or backmutation or both.

The antibodies of the present invention may be isolated antibodies. An “isolated antibody,” as used herein, includes an antibody that is substantially free of other antibodies having different antigenic specificities. Moreover, an isolated antibody may be substantially free of other cellular material and/or chemicals.

Intact antibodies include heteromultimeric glycoproteins comprising at least two heavy and two light chains. Aside from IgM, intact antibodies are usually heterotetrameric glycoproteins of approximately 150 Kda, composed of two identical light (L) chains and two identical heavy (H) chains. Typically, each light chain is linked to a heavy chain by one covalent disulfide bond while the number of disulfide linkages between the heavy chains of different immunoglobulin isotypes varies. Each heavy and light chain also has intrachain disulfide bridges. Each heavy chain has at one end a variable domain (V_{H}) followed by a number of constant regions. Each light chain has a variable domain (V_{L}) and a constant region at its other end; the constant region of the light chain is aligned with the first constant region of the heavy chain and the light chain variable domain is aligned with the variable domain of the heavy chain. The light chains of antibodies from most vertebrate species can be assigned to one of two types called Kappa and Lambda based on the amino acid sequence of the constant region. Depending on the amino acid sequence of the constant region of their heavy chains, human antibodies can be assigned to five different classes, IgA, IgD, IgE, IgG and IgM. IgG and IgA can be further subdivided into subclasses, IgG1, IgG2, IgG3 and IgG4; and IgA1 and IgA2. Species variants exist with mouse and rat having at least IgG2a, IgG2b. The variable domain of the antibody confers binding specificity upon the antibody with certain regions displaying particular variability called complementarity determining regions (CDRs). The more conserved portions of the variable region are called Framework regions (FR). The variable domains of intact heavy and light chains each comprise four FR connected by three CDRs. The CDRs in each chain are held together in close proximity by the FR regions and with the CDRs from the other chain contribute to the formation of the antigen binding site of antibodies. The constant regions are not directly involved in the binding of the antibody to the antigen but exhibit various effector functions such as participation in antibody dependent cell-mediated cytotoxicity (ADCC), phagocytosis via binding to Fcy receptor, half-life/clearance rate via neonatal Fc receptor (FcRn) and complement dependent cytotoxicity via the Clq component of the complement cascade.

The technique of affinity maturation (Marks; Bio/technol 10,779-783 (1992)) may be used to improve binding affinity wherein the affinity of the primary human antibody is improved by sequentially replacing the H and L chain V regions with naturally occurring variants and selecting on the basis of improved binding affinities. Variants of this technique such as “epitope imprinting” are now also available see WO 93/06213. See also Waterhouse; Nucl.Acids Res 21, 2265-2266 (1993).

In certain embodiments, the antigen binding proteins of the present invention have an affinity of at least about 5×10⁴ liter/mole, 1×10⁵ liter/mole, 5×10⁵ liter/mole, or 1×10⁶ liter/mole as measured by an association constant (Ka). In another embodiment, the antigen binding proteins of the present invention binds to a neutralizing epitope of human ADAMTS5 with a dissociation constant (Kd) of less than about 5×10⁻⁴ liter/second, 1×10⁻⁵ liter/second, 5×10⁻⁵ liter/second, or 1×10⁻⁶ liter/second.

The antigen binding protein of the present invention can be characterized by a dissociation constant equal or less than about 9.0×10⁻⁹ M for human ADAMTS5, in some instances it is less than or equal to about 2.5×10⁻¹⁰ M. Antigen binding protein affinity for a target such as human ADAMTS5 can be measured by surface plasmon resonance such as but not limited to BIACORE or Octet. BIAcore kinetic analysis can be used to determine the binding on and off rates of antibodies or fragments thereof to a ADAMTS5 antigen. BlAcore kinetic analysis comprises analyzing the binding and dissociation of a ADAMTS5 antigen from chips with immobilized antibodies or fragments thereof on their surface (see the Example section infra)

The present invention also provides antigen binding proteins that block and/or reduce at least one activity ADAMTS5. In some instances, the antigen binding proteins of the present invention blocks and/or reduces the cleavage of aggrecan by ADAMTS5 at the Glu³⁷³-Ala³⁷⁴ cleavage site. In some aspects, the antigen binding proteins of the present invention are capable of penetrating cartilage, even when administered by a non-articular route of administration. For instance, the antigen binding proteins of the present invention may be administered intravenously, intramuscularly, intraarticularly, subcutaneously, orally, intranasally, and/or by peritoneal administration.

Also provided in the present invention are isolated polynucleotides encoding an antigen binding protein of this invention.

In one embodiment the isolated polynucleotide encodes an antigen binding protein or a fragment thereof comprising an antibody V_{H} domain comprising an amino acid sequence selected from the group consisting of SEQ ID NO: 76, 80, 116, 118, 120, 122, 124, 126, 128, 136, 138, 140, 142, and 144. In one embodiment the isolated polynucleotide is selected from the group consisting of SEQ ID NO: 75, 79, 115, 117, 119, 121, 123, 125, 127, 135, 137, 139, 141, 143, and 159. In one embodiment the polypeptide is an antibody produced from a cell expressing a polynucleotide selected from the group consisting of SEQ ID NO: 75, 79, 115, 117, 119, 121, 123, 125, 127, 135, 137, 139, 141, 143, and 159.

In one embodiment the isolated polynucleotide encodes an antigen binding protein or a fragment thereof comprising an antibody V_{L} domain comprising an amino acid sequence selected from the group consisting of SEQ ID NO: 78, 82, 130, 132, 134, and 146. In one embodiment the isolated polynucleotide is selected from the group consisting of SEQ ID NO: 77, 81, 129, 131, 133, and 145. In one embodiment the polypeptide is an antibody produced from a cell expressing a polynucleotide selected from the group consisting of SEQ ID NO: 77, 81, 129, 131, 133, and 145.

In one embodiment the isolated polynucleotide encodes an antigen binding protein or a fragment thereof comprising an antibody heavy chain comprising an amino acid sequence selected from the group consisting of SEQ ID NO: 68, 72, 84, 86, 88, 90, 92, 94, 96, 104, 106, 108, 110, 112, and 158. In one embodiment the isolated polynucleotide is selected from the group consisting of SEQ ID NO: 67, 71, 83, 85, 87, 89, 91, 93, 95, 103, 105, 107, 109, 111, and 159. In one embodiment the polypeptide is an antibody produced from a cell expressing a polynucleotide selected from the group consisting of SEQ ID NO: 67, 71, 83, 85, 87, 89, 91, 93, 95, 103, 105, 107, 109, 111, and 159.

In one embodiment the isolated polynucleotide encodes an antigen binding protein or a fragment thereof comprising an antibody light chain comprising an amino acid sequence selected from the group consisting of SEQ ID NO: 70, 74, 98, 100, 102, and 114. In one embodiment the isolated polynucleotide is selected from the group consisting of SEQ ID NO: 69, 73, 97, 99, 101, and 115. In one embodiment the polypeptide is an antibody produced from a cell expressing a polynucleotide selected from the group consisting of SEQ ID NO: 69, 73, 97, 99, 101, and 115.

TABLE 1
anti-ADAMTS5 mAb CDR sequence alignment

V_{H} CDR Alignments

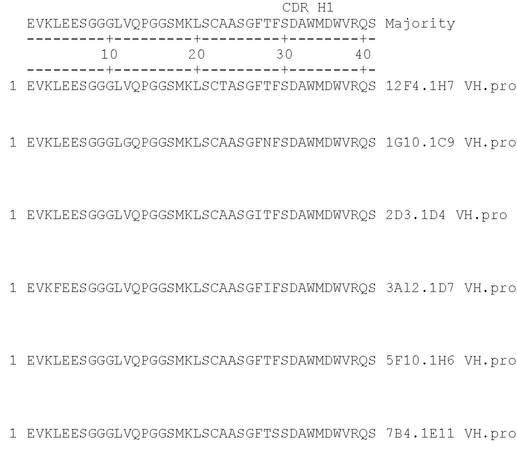

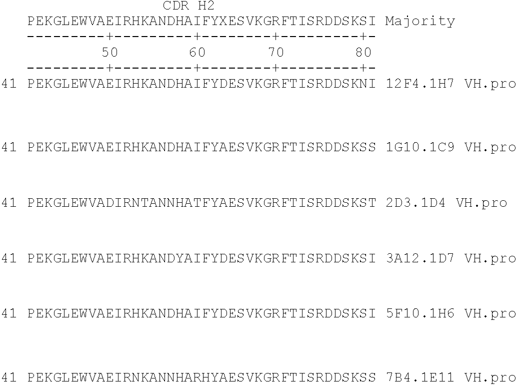

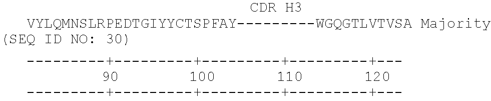

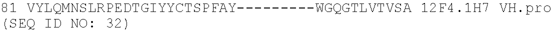

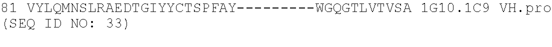

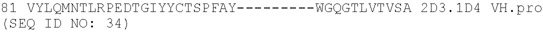

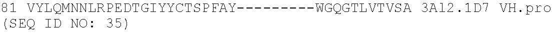

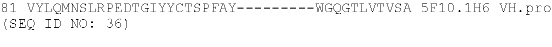

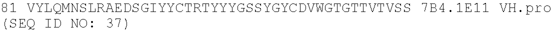

V_{L} CDR Alignments

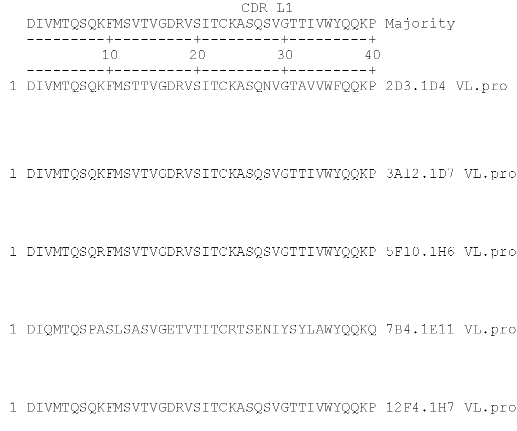

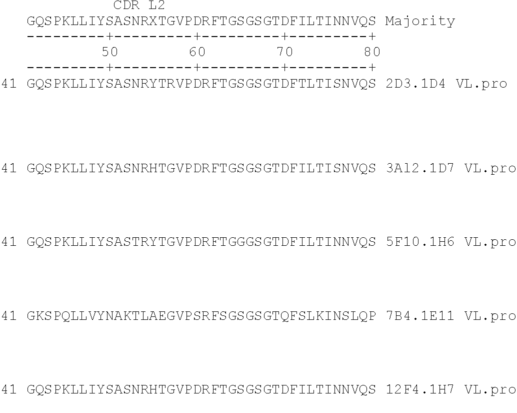

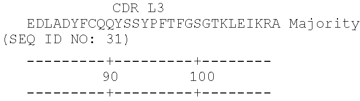

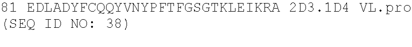

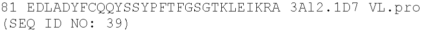

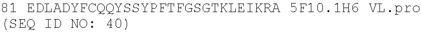

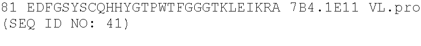

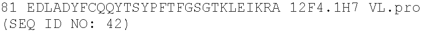

TABLE 2
Antibody variants

| | | | SEQ ID NO: | SEQ ID NO: |
|---|---|---|---|---|
| Antibody | Alternative | | of nucleotide | of amino acid |
| ID | Names | Description | sequence | sequence |
| BPC1622 | 7B4 | 7B4 | 67 | 68 |
| | Chimera | chimeric | | |
| | | heavy chain | | |
| | | 7B4 | 69 | 70 |
| | | chimeric | | |
| | | light chain | | |
| BPC1623 | 12F4 | 12F4 | 71 | 72 |
| | Chimera | chimeric | | |
| | | heavy chain | | |
| | | 12F4 | 73 | 74 |
| | | chimeric | | |
| | | light chain | | |
| BPC1634 | 7B4 H0L0 | 7B4 H0 | 83 | 84 |
| | | heavy chain | | |
| | | 7B4 L0 | 97 | 98 |
| | | light chain | | |
| BPC1635 | 7B4 H1L0 | 7B4 H1 | 85 | 86 |
| | | heavy chain | | |
| | | 7B4 L0 | 97 | 98 |
| | | light chain | | |
| BPC1636 | 7B4 H2L0 | 7B4 H2 | 87 | 88 |
| | | heavy chain | | |
| | | 7B4 L0 | 97 | 98 |
| | | light chain | | |
| BPC1637 | 7B4 H3L0 | 7B4 H3 | 89 | 90 |
| | | heavy chain | | |
| | | 7B4 L0 | 97 | 98 |
| | | light chain | | |
| BPC1638 | 7B4 H4L0 | 7B4 H4 | 91 | 92 |
| | | heavy chain | | |
| | | 7B4 L0 | 97 | 98 |
| | | light chain | | |
| BPC1639 | 7B4 H5L0 | 7B4 H5 | 93 | 94 |
| | | heavy chain | | |
| | | 7B4 L0 | 97 | 98 |
| | | light chain | | |
| BPC1640 | 7B4 H6L0 | 7B4 H6 | 95 | 96 |
| | | heavy chain | | |
| | | 7B4 L0 | 97 | 98 |
| | | light chain | | |
| BPC1641 | 7B4 H0L1 | 7B4 H0 | 83 | 84 |
| | | heavy chain | | |
| | | 7B4 L1 | 99 | 100 |
| | | light chain | | |
| BPC1642 | 7B4 H1L1 | 7B4 H1 | 85 | 86 |
| | | heavy chain | | |
| | | 7B4 L1 | 99 | 100 |
| | | light chain | | |
| BPC1643 | 7B4 H2L1 | 7B4 H2 | 87 | 88 |
| | | heavy chain | | |
| | | 7B4 L1 | 99 | 100 |
| | | light chain | | |
| BPC1644 | 7B4 H3L1 | 7B4 H3 | 89 | 90 |
| | | heavy chain | | |
| | | 7B4 L1 | 99 | 100 |
| | | light chain | | |
| BPC1645 | 7B4 H4L1 | 7B4 H4 | 91 | 92 |
| | | heavy chain | | |
| | | 7B4 L1 | 99 | 100 |
| | | light chain | | |
| BPC1646 | 7B4 H5L1 | 7B4 H5 | 93 | 94 |
| | | heavy chain | | |
| | | 7B4 L1 | 99 | 100 |
| | | light chain | | |
| BPC1647 | 7B4 H6L1 | 7B4 H6 | 95 | 96 |
| | | heavy chain | | |
| | | 7B4 L1 | 99 | 100 |
| | | light chain | | |
| BPC1648 | 7B4 H0L2 | 7B4 H0 | 83 | 84 |
| | | heavy chain | | |
| | | 7B4 L2 | 101 | 102 |
| | | light chain | | |
| BPC1649 | 7B4 H1L2 | 7B4 H1 | 85 | 86 |
| | | heavy chain | | |
| | | 7B4 L2 | 101 | 102 |
| | | light chain | | |
| BPC1650 | 7B4 H2L2 | 7B4 H2 | 87 | 88 |
| | | heavy chain | | |
| | | 7B4 L2 | 101 | 102 |
| | | light chain | | |
| BPC1651 | 7B4 H3L2 | 7B4 H3 | 89 | 90 |
| | | heavy chain | | |
| | | 7B4 L2 | 101 | 102 |
| | | light chain | | |
| BPC1652 | 7B4 H4L2 | 7B4 H4 | 91 | 92 |
| | | heavy chain | | |
| | | 7B4 L2 | 101 | 102 |
| | | light chain | | |
| BPC1653 | 7B4 H5L2 | 7B4 H5 | 93 | 94 |
| | | heavy chain | | |
| | | 7B4 L2 | 101 | 102 |
| | | light chain | | |
| BPC1654 | 7B4 H6L2 | 7B4 H6 | 95 | 96 |
| | | heavy chain | | |
| | | 7B4 L2 | 101 | 102 |
| | | light chain | | |
| BPC1655 | 12F4 H0L0 | 12F4 H0 | 103 | 104 |
| | | heavy chain | | |
| | | 12F4 L0 | 113 | 114 |
| | | light chain | | |
| BPC1656 | 12F4 H1L0 | 12F4 H1 | 105 | 106 |
| | | heavy chain | | |
| | | 12F4 L0 | 113 | 114 |
| | | light chain | | |
| BPC1657 | 12F4 H2L0 | 12F4 H2 | 107 | 108 |
| | | heavy chain | | |
| | | 12F4 L0 | 113 | 114 |
| | | light chain | | |
| BPC1658 | 12F4 H3L0 | 12F4 H3 | 109 | 110 |
| | | heavy chain | | |
| | | 12F4 L0 | 113 | 114 |
| | | light chain | | |
| BPC1659 | 12F4 H4L0 | 12F4 H4 | 111 | 112 |
| | | heavy chain | | |
| | | 12F4 L0 | 113 | 114 |
| | | light chain | | |

TABLE 3

| | Sequence identifier (SEQ ID NO) | |
|---|---|---|
| | amino acid | DNA |
| Sequence Descriptions | sequence | sequence |
| Signal peptide sequence | 46 | 45 |
| 7B4 mouse variable heavy | 48 | 47 |
| 7B4 mouse variable light | 50 | 49 |
| 12F4 mouse variable heavy | 52 | 51 |
| 12F4 mouse variable light | 54 | 53 |
| 7B4 CDRH1 | 55 | |
| 7B4 CDRH2 | 56 | |
| 7B4 CDRH3 | 57 | |
| 7B4 CDRL1 | 58 | |
| 7B4 CDRL2 | 59 | |
| 7B4 CDRL3 | 60 | |
| 12F4 CDRH1 | 61 | |
| 12F4 CDRH2 | 62 | |
| 12F4 CDRH3 | 63 | |
| 12F4 CDRL1 | 64 | |
| 12F4 CDRL2 | 65 | |
| 12F4 CDRL3 | 66 | |
| 7B4 chimera heavy chain | 68 | 67 |
| 7B4 chimera light chain | 70 | 69 |
| 12F4 chimera heavy chain | 72 | 71 |
| 12F4 chimera light chain | 74 | 73 |
| 7B4 chimera heavy chain | 76 | 75 |
| variable region | | |
| 7B4 chimera light chain | 78 | 77 |
| variable region | | |
| 12F4 chimera heavy chain | 80 | 79 |
| variable region | | |
| 12F4 chimera light chain | 82 | 81 |
| variable region | | |
| 7B4 H0 heavy chain | 84 | 83 |
| 7B4 H1 heavy chain | 86 | 85 |
| 7B4 H2 heavy chain | 88 | 87 |
| 7B4 H3 heavy chain | 90 | 89 |
| 7B4 H4 heavy chain | 92 | 91 |
| 7B4 H5 heavy chain | 94 | 93 |
| 7B4 H6 heavy chain | 96 | 95 |
| 7B4 L0 light chain | 98 | 97 |
| 7B4 L1 light chain | 100 | 99 |
| 7B4 L2 light chain | 102 | 101 |
| 12F4 H0 heavy chain | 104 | 103 |
| 12F4 H1 heavy chain | 106 | 105 |
| 12F4 H2 heavy chain | 108 | 107 |
| 12F4 H3 heavy chain | 110 | 109 |
| 12F4 H4 heavy chain | 112 | 111 |
| 12F4 L0 light chain | 114 | 113 |
| 7B4 H0 heavy chain | 116 | 115 |
| variable region | | |
| 7B4 H1 heavy chain | 118 | 117 |
| variable region | | |
| 7B4 H2 heavy chain | 120 | 119 |
| variable region | | |
| 7B4 H3 heavy chain | 122 | 121 |
| variable region | | |
| 7B4 H4 heavy chain | 124 | 123 |
| variable region | | |
| 7B4 H5 heavy chain | 126 | 125 |
| variable region | | |
| 7B4 H6 heavy chain | 128 | 127 |
| variable region | | |
| 7B4 L0 light chain | 130 | 129 |
| variable region | | |
| 7B4 L1 light chain | 132 | 131 |
| variable region | | |
| 7B4 L2 light chain | 134 | 133 |
| variable region | | |
| 12F4 H0 heavy chain | 136 | 135 |
| variable region | | |
| 12F4 H1 heavy chain | 138 | 137 |
| variable region | | |
| 12F4 H2 heavy chain | 140 | 139 |
| variable region | | |
| 12F4 H3 heavy chain | 142 | 141 |
| variable region | | |
| 12F4 H4 heavy chain | 144 | 143 |
| variable region | | |
| 12F4 L0 light chain | 146 | 145 |
| variable region | | |
| 12F4.1H7 heavy chain | 32 | 147 |
| variable region | | |
| 1G10.1C9 heavy chain | 33 | 157 |
| variable region | | |
| 2D3.1D4 heavy chain | 34 | 151 |
| variable region | | |
| 3A12.1D7 heavy chain | 35 | 153 |
| variable region | | |
| 5F10.1H6 heavy chain | 36 | 155 |
| variable region | | |
| 7B4.1E11 heavy chain | 37 | 149 |
| variable region | | |
| 2D3.1D4 light chain | 38 | 152 |
| variable region | | |
| 3A12.1D7 light chain | 39 | 154 |
| variable region | | |
| 5F10.1H6 light chain | 40 | 156 |
| variable region | | |
| 7B4.1E11 light chain | 41 | 150 |
| variable region | | |
| 12F4.1H7 light chain | 42 | 148 |
| variable region | | |
| 12F4 H4L0 IgG1m(AA) heavy chain | 158 | 159 |

As used herein, “patient” refers to a human or other non-human animal.

As used herein, “treatment” means: (1) the amelioration or prevention of the condition being treated or one or more of the biological manifestations of the condition being treated, (2) the interference with (a) one or more points in the biological cascade that leads to or is responsible for the condition being treated or (b) one or more of the biological manifestations of the condition being treated, or (3) the alleviation of one or more of the symptoms or effects associated with the condition being treated. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof.

As used herein, “safe and effective amount” means an amount of at least one antigen binding protein sufficient to significantly induce a positive modification in the condition to be treated but low enough to avoid serious side effects (at a reasonable benefit/risk ratio) within the scope of sound medical judgment. A safe and effective amount of at least one antigen binding protein of the invention will vary with the particular compound chosen (e.g. consider the potency, efficacy, and half-life of the compound); the route of administration chosen; the condition being treated; the severity of the condition being treated; the age, size, weight, and physical condition of the patient being treated; the medical history of the patient to be treated; the duration of the treatment; the nature of concurrent therapy; the desired therapeutic effect; and like factors, but can nevertheless be routinely determined by the skilled artisan.

The antigen binding proteins of the invention may be administered by any suitable route of administration, including both systemic administration and topical administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, and subcutaneous injection or infusion, including intraarticular administration. Inhalation refers to administration into the patient's lungs whether inhaled through the mouth or through the nasal passages. Topical administration includes application to the skin as well as intraocular, otic, intravaginal, and intranasal administration.

The antigen binding proteins of the invention may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a antigen binding protein of the invention depend on the pharmacokinetic properties of that compound, such as absorption, distribution, and half-life, which can be determined by the skilled artisan. In addition, suitable dosing regimens, including the duration such regimens are administered, for a compound of the invention depend on the condition being treated, the severity of the condition being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change.

In certain embodiments the antibody is used to deliver a drug to the cartilage. Such a drug could be an aggrecanase inhibitor, an anti-inflammatory drug, steroid or a drug related to pain management. Accordingly, in one aspect the invention is a method of delivering a drug to cartilage comprising lining the drug to an antibody of the present invention. Such delivery can be conducted in vitro, ex vivo, or in vivo.

In another embodiment the antibody is used to deliver a growth factor to the cartilage which would promote the growth of new cartilage. Such growth factors include Bone Morphogenic proteins, particularly BMP-7. Such delivery can be conducted in vitro, ex vivo, or in vivo.

### EXAMPLES

The following examples illustrate various aspects of this invention.

### Example 1

### Human OA Cartilage Explant Protocol

This biomarker assay should identify patient subsets within the OA disease spectrum that have elevated aggrecanase activity and will be the most suitable candidates for treatment with aggrecanase inhibitors. Correlations may also be drawn from relationships between the ARGSVIL neoepitope and other study markers and endpoints to enable more effective patient stratification and disease characterization. Human tissue (femoral chondytes and tibial plateau) was received on ice within 24 hours of removal. See Table 4.

TABLE 4
Human OA Total Knee Replacement Donor Information

| Donor | Age | Gender | Diagnosis | Procedure | Comorbidities | Medications |
|---|---|---|---|---|---|---|
| 62672 | 69 | F | OA | TKA | HTN, depression, lumar disk disease | Verapamil, Vaseretic, Zocor, Diclofenac- |
| | | | | | | Misoprostol, Prozac, Darvocet, Desyrel, Lidocaine, |
| | | | | | | Tetracaine |
| 62671 | 61 | M | OA | TKA | Hypercholesterolemia, HTN, GERD, Seizure | Hyzaar, Lipitor, Carbatrol, Omega 3, |
| | | | | | disorder, Lumar disk disease | Cholecalciferol |
| 35286 | 60 | M | OA | Left TKA | HTN, Hypercholesterolemia, Sleep Apnea, | Cozaar, Zocor, Prozac, Tenormin, Glucophage, |
| | | | | | Diabetes type II, Stress, incontinence, Breast | Starlix, Fergon, Cholecalciferol |
| | | | | | Cancer, Depression | |
| 35275 | 59 | M | OA | Bilateral | HTN, Asthma, Vision Deficit, Renal Calculus | Choline, Omacor, Lovaza, Motrin, Benicar HCT, |
| | | | | TKA | | Cardizem CD |
| 35274 | 69 | F | OA | Right | HTN, Dysrhythmia, GERD, Anxiety Disorder | Lasix, Toprolex, Protonix, Percocet, Oxycontin, |
| | | | | TKA | | Celebrex, Pseudoephedrine HCL |
| 34211 | 55 | F | OA | Left TKA | HTN, Asthma, Ulcers, GERD, Urinary | Advair, proventil, protonix, triamterene,/HCTZ, |
| | | | | | incontinence, meningiom of cranium, carpal | Azor, Soliferacin, Fergon, . . . ndamycin, Tylenol 3 |
| | | | | | tunnel, rotator cuff injury | |
| 34210 | 63 | M | OA | Bilateral | HTN, Diabetes | Avapro, Metormin |
| | | | | TKA | | |
| 34021a | 62 | F | OA | Left TKA | HTN | Zetia, Toprol, Lipitor, ASA |
| 34020a | 55 | F | OA | Left TKA | HTN, Obesity | Lisi..pril, HCTZ |
| 33833 | 69 | F | OA | Right | HTN, MVP, Hypothyroid | ASA, Synthroid, Toprol, Floic Acid, Calcium |
| | | | | TKA | | |
| 33748 | 76 | F | OA | Right | HTN, Dysrhythmia, Celiac Sprue, H. Pylori, | Indium, Allegra, Fergon |
| | | | | TKA | Depression, Anxiety, Vertigo | |
| 33747 | 61 | F | OA | Right | Interstitial Cystitis, Fibroids, Anxiety | Fergon, Calcium |
| | | | | TKA | | |
| 33746 | 93 | M | OA | Right | HTN, Goiter, GERD, Depression | Altace, Hydrodlull, Nexium, Lipitor, Celebrex, |
| | | | | TKA | | Ketoconazole, Fluticasone |
| 33745 | 76 | F | OA | Right | Hypercholesterolemia, Hypothyroidism, | Clartin, Prevacid, Duragesic patch, Valium, |
| | | | | TKA | GERD, Anxiety, Depression, Migranes, | Resteril, Levsin, Quinine Sulfate, Lidocaine, Elavil, |
| | | | | | Squamous Cell Carcinoma-no chemo/rad. | Mobic, Zenaflex, Topamax, Synthroid |

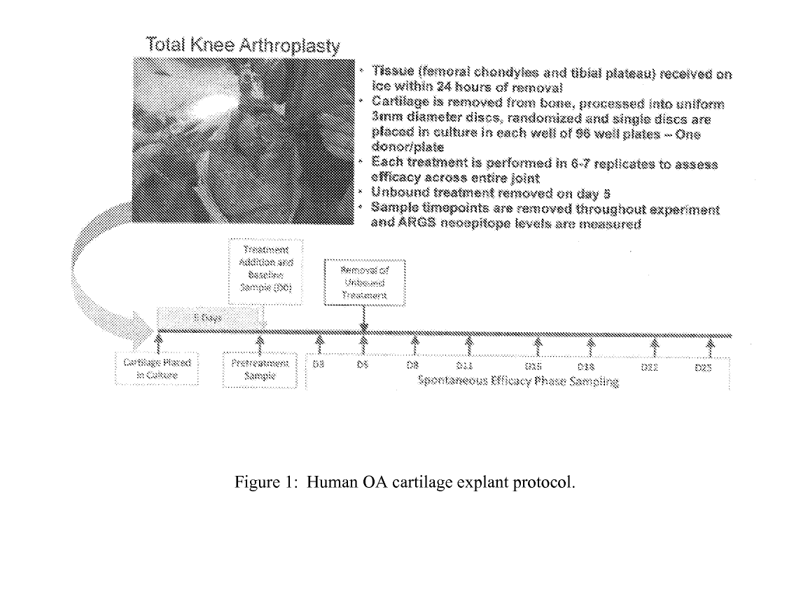

The cartilage was removed from bone, processed into uniform 3mm diameter discs. Randomized and single discs are placed in culture in each well of 96 well plates (one tissue donor/plate) and cultured for five days in DMEM+10% FCS and antibiotics. At day 5 the wells are treated with 200 ug/mL 12F4.H4L0 antibody for five days. Each treatment was performed in 6-7 replicates to assess efficacy across entire joint. After five days any unbound 12F4.H4L0 was removed. ARGSVIL neopeptide levels are measured at days 8, 11, 15, 18, 22, 25 post treatment. See FIG. 1.

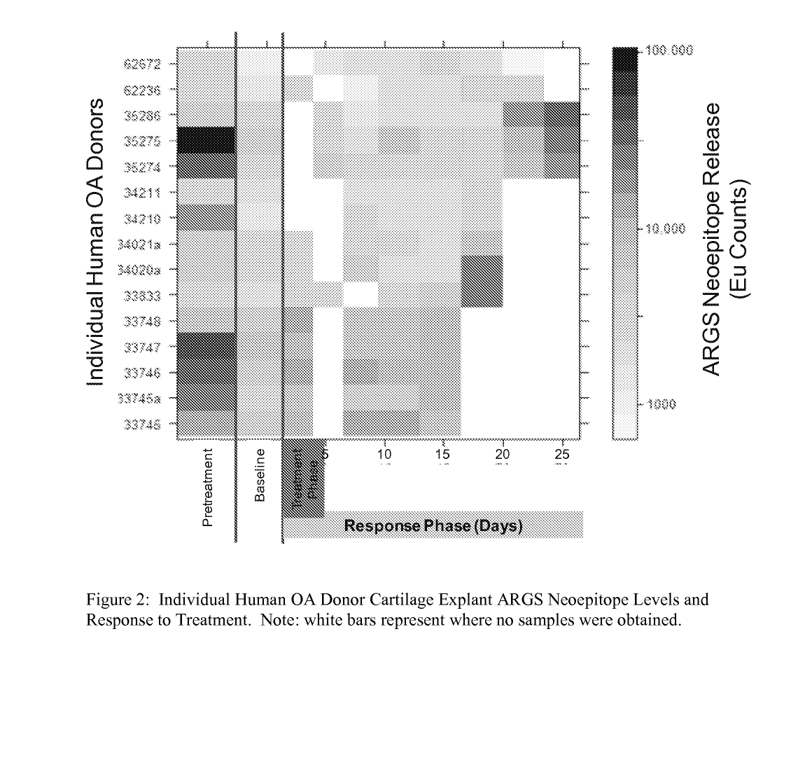

FIG. 2 is a schematic showing the amount of ARGS neopeptide found in each individual donor's cultured cartilage at different time points. The donor identification is found on the left Y-axis. The number of days pre and post-treatment is found on the X-axis. The amount of ARGS neopeptide is expressed by shading (see right Y-axis).

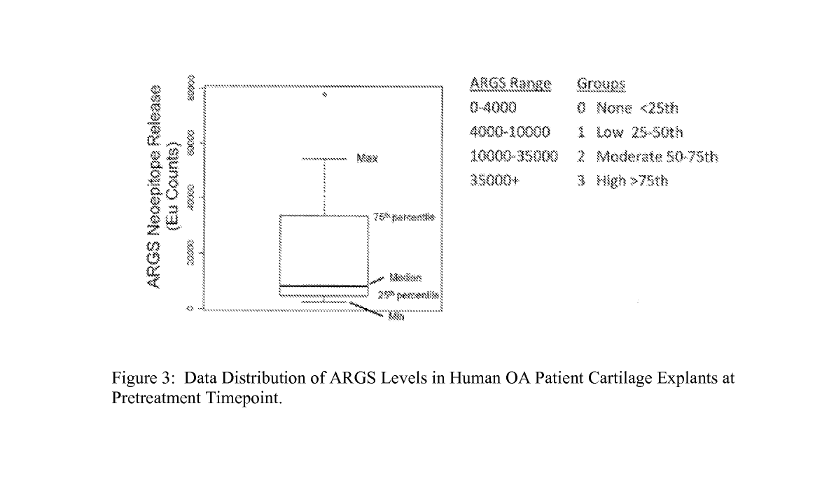

FIG. 3 is a Box Whisker Plot of ARGS neopeptide levels before treatment with 12F4.H4L0. The plot displays the 25th-75th percentiles in the boxed area and the “whiskers” show the minimum and maximum of the data. The median value is depicted by the horizontal line. The donors are divided into four groups (0-3) based on pre-treatment levels.

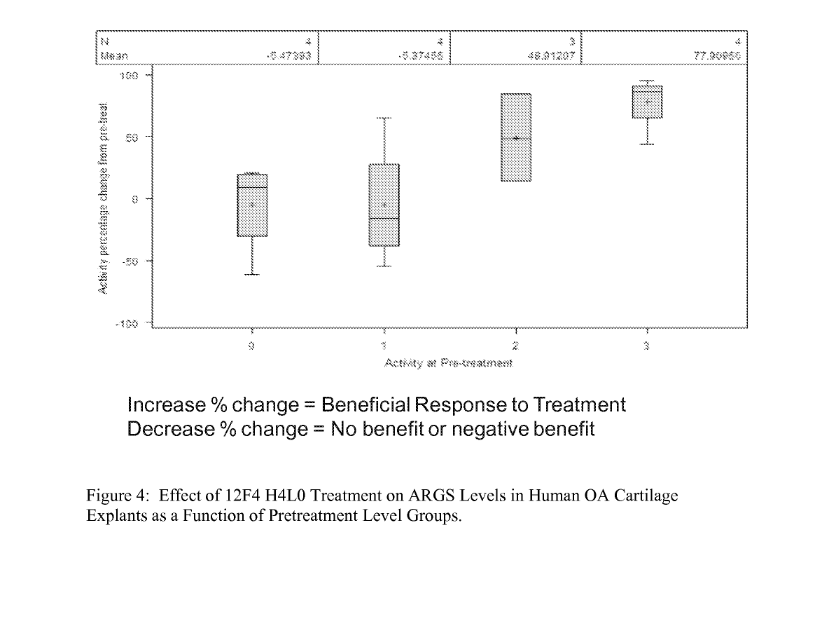

After dividing the donors into the four pre-treatment groups (0-3), all of the post-treatment neopeptides levels for each donor were averaged. These averaged values were then presented in another Box Whisker plot that shows the 25^{th}-75^{th} percentiles in the boxed area and the “whiskers” showing the minimum and maximum of the data. The median value is depicted by the horizontal line within the box and the “+” is the average. See FIG. 4.

Based on pre and post treatment ARGS neopeptide levels, the donors are stratified into moderate to high and low pretreatment levels as well as moderate to high and low post-treatment response levels. Statistical analysis show that ARGS neopeptide pre-treatment levels can be a significant predictor of ARGS neopeptide levels post-treatment with an aggrecanase inhibitor. These results are summarized in Table 5.

TABLE 5
Analysis Summary

| | |
|---|---|
| Donors with Moderate to High Pretreatment Disease | 47% (7 of 15) |
| Burden (Predicted Trial Inclusion Rate) | |
| Donors with None to Low Pretreatment Disease Burden | 53% (8 of 15) |
| (Predicted Trial Exclusion Rate) | |
| Correlation of Pretreatment Disease Burden to | |
| Response (Predictive value of treatment) | |
| After treatment with 12F4 | |
| Low ARGS to Low Anti-ADAMTS5 response (TN) | 88% (7 of 8) |
| Mod to High ARGS to Mod to High Response (TP) | 86% (6 of 7) |
| Total | 87% (13/15) |
| False negative/false positive | 14%/12% |
| After treatment with Isotype | |
| Mod to High ARGS to low or negative Response | 100% (2 of 2) |

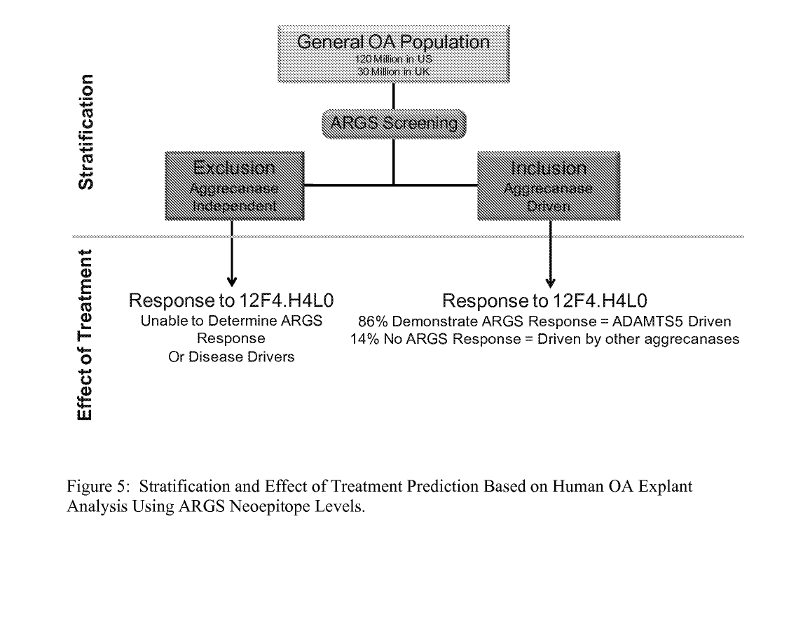

FIG. 5 is a schematic summarizing how patients are stratified using ARGS neopeptide pre-treatment levels and how patients with moderate to high levels of ARGS neopeptide pre-treatment levels are more responsive to aggrecanase inhibitor-based therapy than patients with low levels of ARGS neopeptide pre-treatment levels.

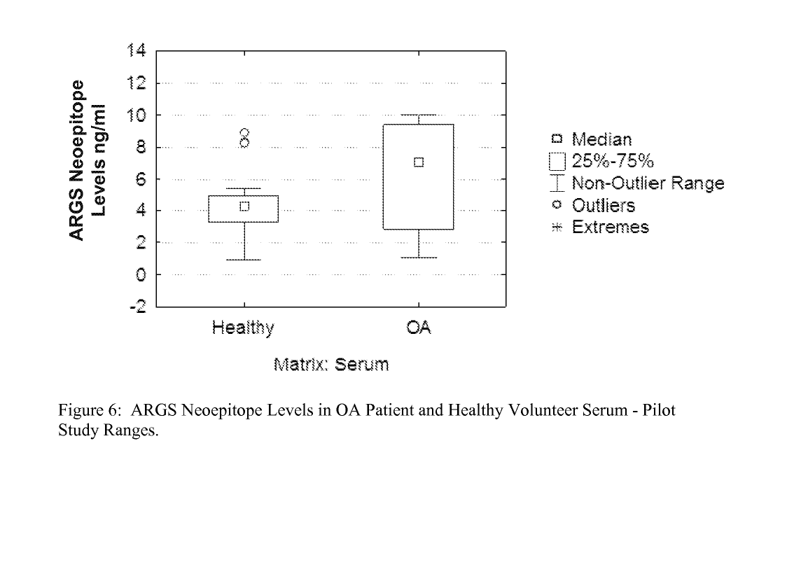

FIG. 6 shows that ARGS neoepitope levels in OA patient and healthy volunteer serum indicate that patients with OA and with moderate to high ARGS neopeptide levels would be good candidates for aggrecanase inhibitor-based therapy.

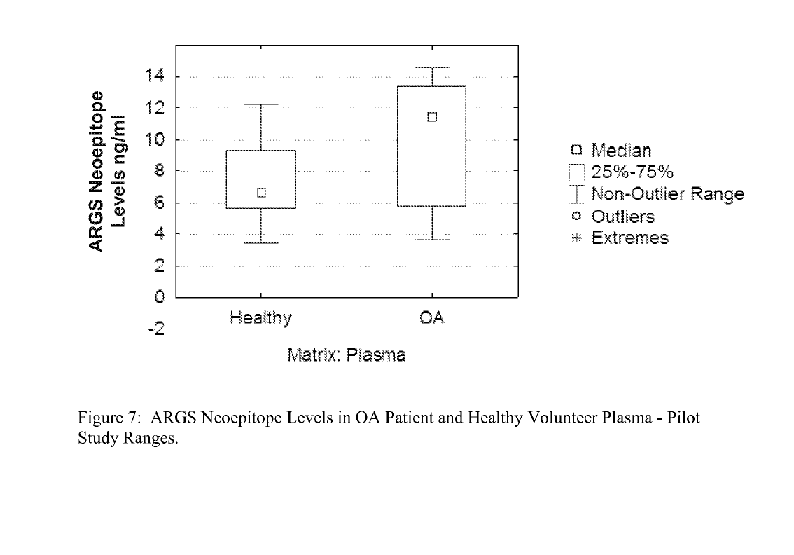

FIG. 7 shows that ARGS neoepitope levels in OA patient and healthy volunteer plasma indicate that patients with OA and with moderate to high ARGS neopeptide levels would be good candidates for aggrecanase inhibitor-based therapy.

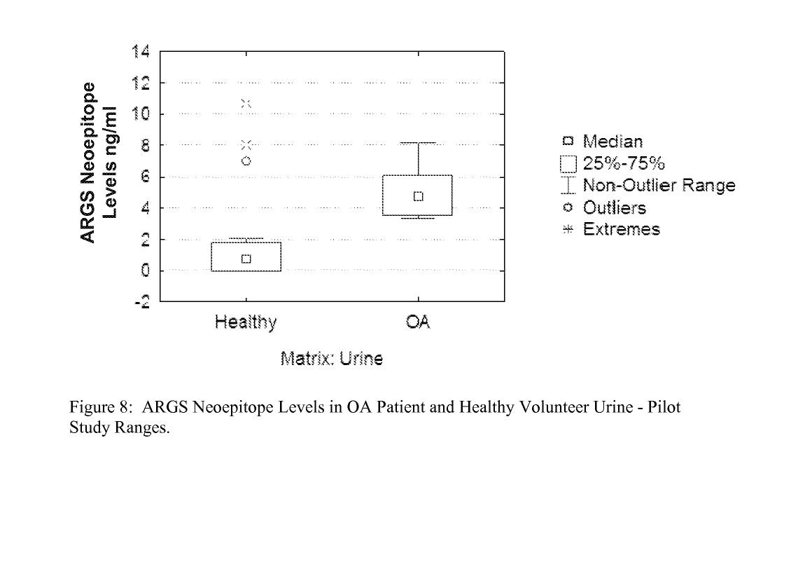

FIG. 8 shows that ARGS neoepitope levels in OA patient and healthy volunteer urine indicate that patients with OA and with moderate to high ARGS neopeptide levels would be good candidates for aggrecanase inhibitor-based therapy.

### Example 2

### Measurement of ARGSVIL in Human Biological Samples

This example describes the procedures required to validate the electrochemilumescent (ECL) immunoassay developed for the measurement of the 374-ARGS neoepitope of aggrecanase-cleaved aggrecan in human serum, plasma, urine and synovial fluid to facilitate the clinical development of the aggrecanase inhibitor, ADAMTS5 mAb for the treatment of Osteoarthritis (OA).

### ABBREVIATIONS

ADAMTS A disintegrin and metalloproteinase with thrombospondin motif

BCC Back calculated concentration

BDU Blood Donation Unit

BT Bench top

C Cycle number

Conc. Concentration

CS Chondroitin Sulphate

CV Coefficient of Variance

ECL Electrochemical Luminescence

FT Freeze and thaw

FTIH First time in human

GSK GlaxoSmithKline

HABR Hyaluronic Acid Binding Region

HD Healthy Donor(s)

KS Keratin Sulfate

LOD Limit of Detection

LLOQ Lower Limit of Quantification

μg Micro-gram

μL Micro-litre

mg Milli-gram

mL Milli-litre

MSD Meso Scale Discovery

NC Negative control

ng Nano-gram

OA Osteoarthritis

pg Pico-gram

PPE Personal Protective Equipment

RE Relative Error

RT Room Temperature

S Sample

Sp Spike

SD Standard Deviation

t Time

ULOQ Upper Limit of Quantification

VC Validation Control

Vol Volume

### OVERVIEW

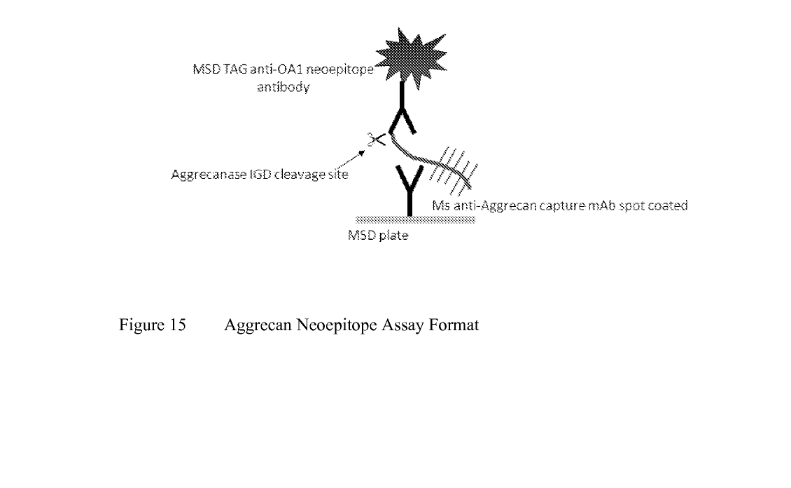

An MSD electrochemiluminescent immunoassay has been developed to measure the 374-ARGS neoepitope (Error! Reference source not found.). The assay uses a commercially available antibody directed against the hyaluoronic-acid binding region (HABR) of aggrecan as the capture antibody. 374-ARGS containing fragments present in human samples are first captured, followed by detection using a sulfo-TAG labelled monoclonal antibody OA-1 that recognizes the 374-ARGS neoepitope sequence. The amount of 374-ARGS neoepitope fragments present in the sample was determined based on a standard curve generated with ADAMTS-5 digested recombinant G1-IGD-G2 aggrecan which was diluted in the appropriate human pooled matrix (plasma, serum, urine or synovial fluid) which has been depleted of endogenous 374-ARGS neoepitope (FIG. 15).

Standard curve generated by incubation of ADAMTS5 enzyme with full length aggrecan.

### MATERIALS/EQUIPMENT

The reagents and equipment used in the assay validation is listed in the assay method. The supplier together with the batch or lot number are specified in the validation report.

All significant items of equipment (i.e. those items which are necessary to reproduce method conditions) used in performing the validation shall be identified in the validation report. The same pieces of equipment are used throughout the validation.

### PROCEDURE

Sample Preparation

Serum Collection

Whole blood was collected into serum separator tubes (SST) and left to coagulate at ambient temperature for at least 30 minutes. After clotting, tubes are centrifuged in a swing bucket centrifuge at 1500×g for 15 minutes at 2-8° C. Serum was harvested using a fine tipped pipette and aliquoted into appropriately labelled polypropylene screw-cap cryotubes. Tubes are frozen at −80° C. until assayed.

Plasma Collection

Whole blood was collected in sodium heparin blood collection tubes and mixed well by inverting tube several times. Tubes are centrifuged within 1 hour of collection at 1500×g for 15 minutes at 2-8° C. Plasma was collected (avoiding disturbance of the pellet or buffy coat) using a fine tipped pipette and aliquoted into appropriately labelled polypropylene screw-cap cryotubes. Tubes are frozen at −80° C. until assayed.

Urine Collection

A minimum of 10 mL per patient was collected into a labelled 120 mL sterile urine filter collection pot or sterile non filter urine collection pot. For samples collected into non filter urine collection pot, invert sample to mix and transfer to appropriate number of centrifuge tubes (15 mL or 50 mL, as appropriate to the urine volume collected) and spin at 600×g for 5 minutes at 2-8° C. within 1 hours of collection. For samples collected into filter urine collection pots, sample was inverted and transferred to storage tubes using urine transfer straw. Samples are aliquoted into appropriately labelled polypropylene screw-cap cryotubes. Tubes are frozen at −80° C. until assayed.

Synovial Fluid Collection

Synovial fluid was obtained using standard procedures and frozen at −80° C. until assay. Given the difficulty in obtaining samples, samples will not be treated with hyaluronidase before freezing.

All samples used for validation are frozen prior to use, unless otherwise stated.

Preparation of Validation Control Samples (VCs)

Validation control samples are prepared over a range of 5 analyte concentrations using 374-ARGS neoepitope depleted pooled human serum. The concentrations used are 800, 200, 50, 10 and 2 ng/mL. The VCs are made up in one batch, aliquoted and stored at −80° C., allowing a sufficient number of aliquots to cover the validation process. Table 1 summarises the preparation of VCs for this assay validation.

Aliquot 75 μl at of each VC in appropriate tubes and store at −80° C. On day of use thaw a set of VCs for each plate, use within 30 minutes of thawing.

TABLE 1
Preparation of VCs

| | Volume of 374- | Volume of | VC |
|---|---|---|---|
| VC | ARGS depleted human | stock solution or | concentration |
| Number | serum (mL) | VC | (ng/mL) |
| 1 | 8000 | 64 μL from | 800 |
| | | 100 μg/mL stock of | |
| | | aggrecan | |
| | | neoepitope | |
| 2 | 6000 | 2000 mL | 200 |
| | | from VC 1 | |
| 3 | 6000 | 2000 mL | 50 |
| | | from VC 2 | |
| 4 | 6400 | 1600 mL | 10 |
| | | from VC 3 | |
| 5 | 6400 | 1600 mL | 2 |
| | | from VC 4 | |

Preparation of Calibration Standards

A 10-point standard curve was prepared in relevant matrix (human serum, plasma, urine or synovial fluid depleted of endogenous 374-ARGS neoepitope), covering the range of expected analyte concentrations, from 1000 ng/mL to 0.15 ng/mL including negative control (blank). As the concentration of aggrecan neoepitope was not known after digest, all concentrations stated are that of the undigested aggrecan. Tables 2 summarises the calibration standard preparation.

TABLE 2
Preparation of aggrecan neoepitope reference standards

| | Volume of | Volume and | Concentration |
|---|---|---|---|
| Identification | matrix | source | (ng/mL) |
| Std 1 | 990 μL | 10 μL of | 1000.00 |
| | | 100 μg/mL stock of | |
| | | aggrecan neoepitope | |
| Std 2 | 100 μL | 50 μL of Std 1 | 333.33 |
| Std 3 | 100 μL | 50 μL of Std 2 | 111.11 |
| Std 4 | 100 μL | 50 μL of Std 3 | 37.04 |
| Std 5 | 100 μL | 50 μL of Std 4 | 12.35 |
| Std 6 | 100 μL | 50 μL of Std 5 | 4.12 |
| Std 7 | 100 μL | 50 μL of Std 6 | 1.37 |
| Std 8 | 100 μL | 100 μL of Std 7 | 0.46 |
| Std 9 | 100 μL | 100 μL of Std 8 | 0.15 |
| Std 10 (NC) | 100 μL | — | 0 |

### VALIDATION PARAMETERS

Standard Curve Precision

To evaluate the precision of the standard curve, the standards together with the negative control sample (blanks—no aggrecan neoepitope) was analysed in duplicate, in 6 independent validation runs (day 1/plates 1&2, day 2/plates 1-3, day 3/plates 1&2, day 4/plates 1&2, day 5/plates 1&2, and day 6/plates 1-4). Standard curve precision was evaluated in each of the matrices of interest: 374-ARGS neoepitope depleted pooled human serum, plasma, urine and synovial fluid.

The quality of the fit of each standard curve to the data should be assessed prior to performing back calculations from that curve. A four parameter logistic curve versus log10 transformed concentrations may be appropriate for fold dilutions. Where assay signal range is large, also consider log10 transformation of signal. If in doubt, seek the advice of a statistician.

The LOD was determined by back calculating the mean signal of the blanks (zero analyte samples)+2SD. The LLOQ was determined by the lowest standard on the curve that results in percentage CV values that are consistently lower than 20% and are above the LOD.

Acceptance criteria:

(1) The back calculated values for a minimum of 75% of the standards should be within ±20% relative error (RE) of the theoretical concentration (±25% RE for samples 1-2×LLOQ and ULOQ) and the percent coefficient of variation (% CV) should be ≦20%.

(2) The cumulative mean of all 6 runs should not exceed ±15% RE and 15% CV for each standard (≦±20% RE and CV for samples 1-2×LLOQ and ULOQ).

Intra and Inter Assay Precision

The precision of the immunoassay was evaluated by determining the % CV for inter- and intra- assay analysis.

5 VC samples are analysed in triplicate on 5 occasions (day 1/plate 1, day 2/plate 1, day 3/plate 1, day 4/plate 1, and day 5/plate 1). Intra and inter assay precision was measured in 374-ARGS neoepitope depleted pooled human serum matrix only.

Acceptance criteria: The precision of intra and inter assay was ≦20% CV (≦25% CV for samples 1-2×LLOQ and ULOQ).

Cross Plate Precision

To monitor any potential assay plate drift, 374-ARGS neoepitope depleted pooled human serum spiked with 50 ng/mL aggrecan neoepitope was added to each well of a 96-well plate and tested once (day 3/plate 3). Cross plate variance was analysed between all the columns and rows including four corners of the 96-well plate.

Acceptance criteria: %CV for the obtained signal values comparing all 96 replicates was ≦10% CV. Mean column values (A1:H1 to A12:H12), mean row values (A1:A12 to H1:H12) and corner-to-corner values (A1, A12, H1 and H12) was ≦20% CV.

Accuracy

The accuracy of the immunoassay was determined by assessing the recovery of 3 freshly spiked concentrations of aggrecan neoepitope (200 ng/mL, 50 ng/mL and 10 ng/mL) into 374-ARGS neoepitope depleted pooled human serum, and tested in triplicates on 3 separate occasions (day 1/plate 1, day 2/plate 1, and day 3/plate 1).

Acceptance criteria: Mean of triplicate back calculated values on each occasion within ≦±20% RE from nominal values (≦±25% RE for samples 1-2×LLOQ and ULOQ).

Assay Specificity

The specificity of the assay for the 374-ARGS neoepitope was determined by assessing the following parameters.

Matrix Effects

The matrix interference of the assay was determined by assessing the recovery of freshly spiked aggrecan neoepitope (50 ng/mL) analyte into 10 healthy native biological serum, plasma, and urine samples (HD) and compared to unspiked samples (day 6/plates 1-3). The spiked and unspiked samples are analysed in duplicate and the % RE for spiked samples calculated using the theoretical concentration (unspiked native sample+50 ng/mL spike).

Acceptance criteria: ≦±20% RE for a minimum of 8/10 samples (≦±25% RE for samples 1-2×LLOQ).

Specificity for the Neoepitope

To assess the potential of the assay to detect 374-ARGS neoepitope sequence in aggrecanase-cleaved aggrecan, an assay standard curve was tested in 374-ARGS neoepitope depleted pooled human serum using the undigested recombinant G1-IGD-G2 aggrecan and compared to the ADAMTS5 digested aggrecan (day 5/plate 2). The % RE for each calibration concentration was calculated and the result documented in the final validation report.

Specificity of the OA-1 antibody for the immunising neoepitope peptide will not be assessed since it has already been investigated in earlier studies (Pratta et al, Osteoarthritis & Cartilage 2006, 14: 702-713).

Evaluation of Sample Stability

To assess freeze/thaw stability the effect of 3 non-accelerated (minimum 12 hours freezing at −80° C.) freeze/thaw cycles (t=1, 2 and 3) are analysed and compared to the fresh sample (t=0) (day 1/plates 1&2, day 2/plates 1-3, day 3/plates 1&2, and day 4/plates 1&2). Sample stability will also be tested at room temperature (t=1.1) and 2-8° C. (t=1.2) for 24 hours (day 1/plates 1&2, and day 2/plates 1-3). 374-ARGS neoepitope depleted pooled human serum, plasma, urine and synovial fluid spiked with 200 and 10 ng/mL aggrecan neoepitope was analysed in duplicate. Also two HD serum, plasma, urine and synovial fluid samples are subject to the same freeze/thaw cycles. For each sample % RE was calculated using t=0 as reference.

Benchmark criteria: Samples are considered stable if the % RE was ≦±20% compared to t=0.

Long term stability was tested outside of this validation, recommended time course was 1 month, 3 months, 6 months and 12 months at −80° C.

Normal and OA 374-ARGS Range

To evaluate the normal range of 374-ARGS neoepitope levels 20 HD matched serum, plasma and urine samples are analysed in duplicate to confirm the range (day 6/plates 1-3). To confirm disease ranges, matched urine, serum, plasma and synovial fluid are analysed from a minimum of 5 OA patients (day 6/plates 1-4). Acceptable results for precision was ≦20% CV. In addition synovial fluid from healthy individuals will also be tested (number tested are dependent on number received under NDRI agreement).

Linearity of Dilution

The linearity of dilution was assessed by diluting a spiked (500 ng/mL of aggrecan neoepitope) HD serum sample 2×, 4×, 8×, 16×, 32×, and 64×, the same HD serum sample will also be diluted unspiked as above. Dilutions are carried out in 374-ARGS neoepitope depleted pooled human serum, and tested in triplicates (day 5/plate 1).

Acceptance criteria: The dilutions are considered linear if mean of the duplicate back-calculated concentrations are within ±20% RE of the nominal concentration after the dilution factor has been applied.

### Example 3

### Additional Measurement of ARGSVIL in Human Biological Samples

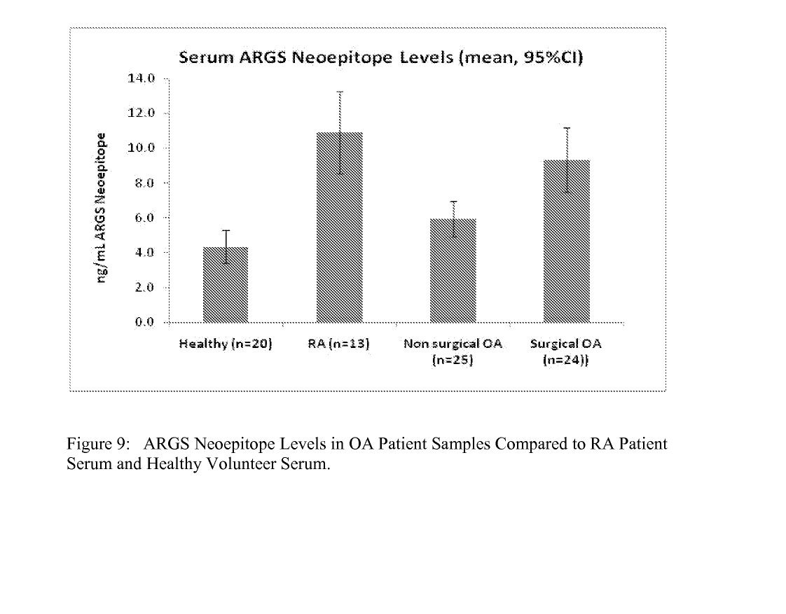

FIG. 9 shows that serum ARGS neoepitope levels are elevated in surgical OA compared to non surgical patient samples and healthy volunteers. ARGS levels in surgical OA patients are similar to ARGS levels in RA patient serum.

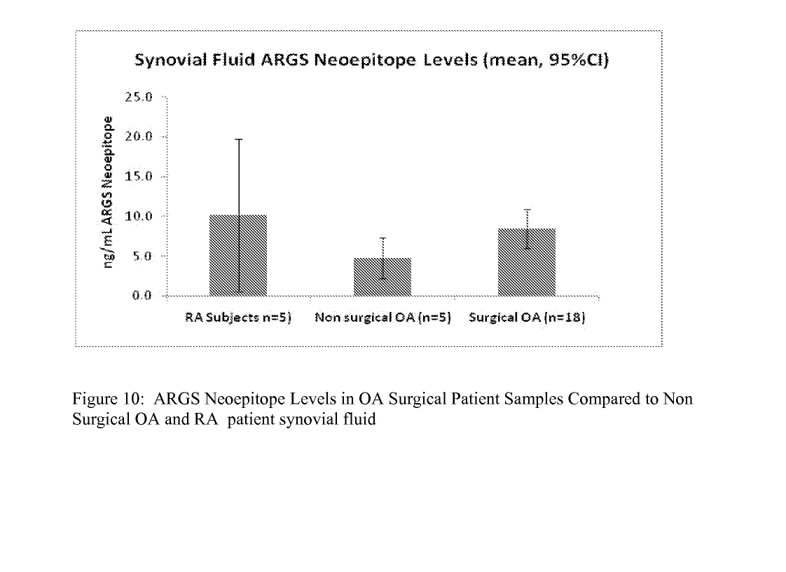

FIG. 10 shows that synovial fluid ARGS neoepitope levels are significantly elevated in samples from surgical OA patients compared to non surgical OA patients. Mean ARGS neoepitope levels are similar in surgical OA patients compared to RA patients.

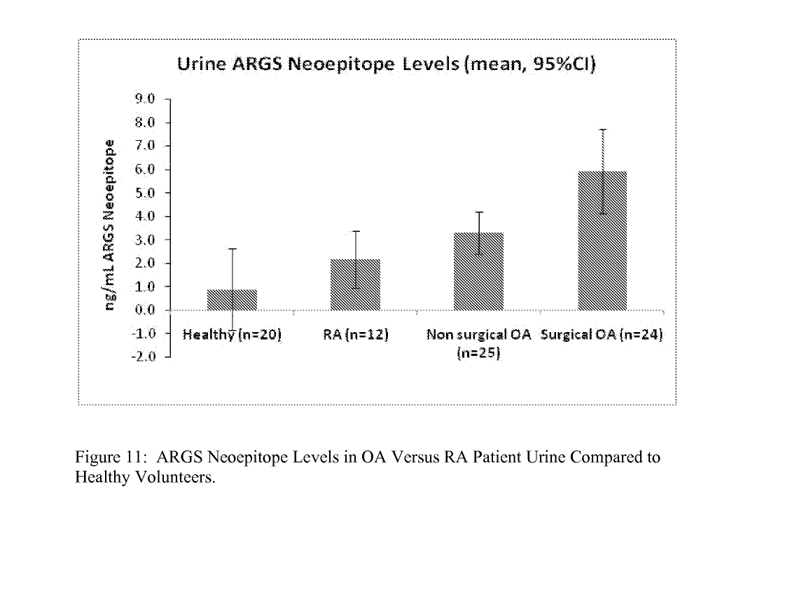

FIG. 11 demonstrates that urine ARGS Neoepitope levels are elevated in samples from surgical OA patients compared to non surgical OA, RA and healthy volunteers.

Intra- and Inter- assay precision—calculation of coefficient of variation within and between assays.

Five serum validation control (VC) samples were analysed in replicates of six on one occasion to evaluate intra-assay precision. Inter-assay precision was determined by analysing five VC samples in triplicate on three occasions. All VC are within ≦20% CV (≦25% CV for samples 1-2×LLOQ and ULOQ).

Plasma standard curves were run over 7 occasions; all samples above LLOQ were within ≦20% CV (≦25% CV for samples 1-2×LLOQ and ULOQ).

Sensitivity—minimal detectable concentration

Lower limit of quantification (LLOQ) was 1.37 ng/mL in serum and plasma, 0.46 ng/mL in urine, and 4.12 ng/mL in synovial fluid.

Specificity—cross-reactivity to contaminants

To assess the potential of the assay to detect ³⁷⁴-ARGS neoepitope sequence in aggrecanase-cleaved aggrecan, an assay standard curve was tested in a ³⁷⁴-ARGS neoepitope depleted pooled human serum using the undigested recombinant G1-IGD-G2 aggrecan and compared to the ADAMTS5 digested aggrecan.

All values were below the LLOQ for the undigested recombinant G1-IGD-G2 aggrecan curve. It can therefore be concluded that undigested recombinant G1-IGD-G2 aggrecan was not detected in this assay and the assay was specific for the ³⁷⁴-ARGS neoepitope.

Effect of freeze thaw of biomarker concentrations (if known)

Validation controls (frozen spiked samples) were stable through 3 freeze/thaw cycles in serum, urine and synovial fluid (within ≦20% RE (≦25% RE for samples 1-2×LLOQ and ULOQ)). Plasma samples were within ≦30% RE.

Stability (At various temperatures including room temp, 4° C., −20° C., −80° C. for various lengths of time)

Validation controls were stable at 2-8° C. and room temperature for 24 hours in serum, plasma, urine and synovial fluid (all samples are considered stable if the % RE was ≦±20% compared to fresh), lower spiked values a little above 20, though ≦±30% RE.

Diurnal Variation

No diurnal variation was seen in serum or urine

The ARGS assay can reliably quantify aggrecanase activity in serum, plasma, urine and synovial fluid (Table 3). In the assay the lower limit of quantification (LLOQ) was reproducibly determined to be 1.37 ng/mL in serum and plasma, 0.46 ng/mL in urine, and 4.12 ng/mL in synovial fluid and the range of the standard curve tested was 0.98 ng/mL-1000 ng/mL.

TABLE 3
Preliminary Data for ARGS levels in matched samples
from healthy human donors (n = 20) and matched
commercial OA donor samples (n = 5)

| Range | | | | | | | |
|---|---|---|---|---|---|---|---|
| Healthy | | | | | | 5th | 95th |
| (n = 20) | Min | Max | mean | SD | median | percentile | percentile |
| serum | 0.94 | 8.89 | 4.34 | 2.17 | 4.30 | 4.30 | 8.35 |
| plasma | 1.42 | 10.26 | 5.36 | 2.49 | 4.65 | 4.65 | 8.38 |
| urine | 0.25 | 10.66 | 2.68 | 3.32 | 1.28 | 4.42 | 9.23 |
| Range OA | | | | | | 5th | 95th |
| (n = 5) | Min | Max | Mean | SD | Median | percentile | percentile |
| serum | 1.06 | 9.99 | 6.07 | 3.97 | 7.09 | 7.09 | 9.87 |
| plasma | 1.62 | 12.55 | 8.76 | 4.93 | 10.44 | 10.44 | 12.38 |
| urine | 3.34 | 8.14 | 5.18 | 1.99 | 4.23 | 4.75 | 7.73 |
| SF | 2.25 | 8.33 | 4.76 | 2.94 | 4.23 | 4.23 | 7.98 |

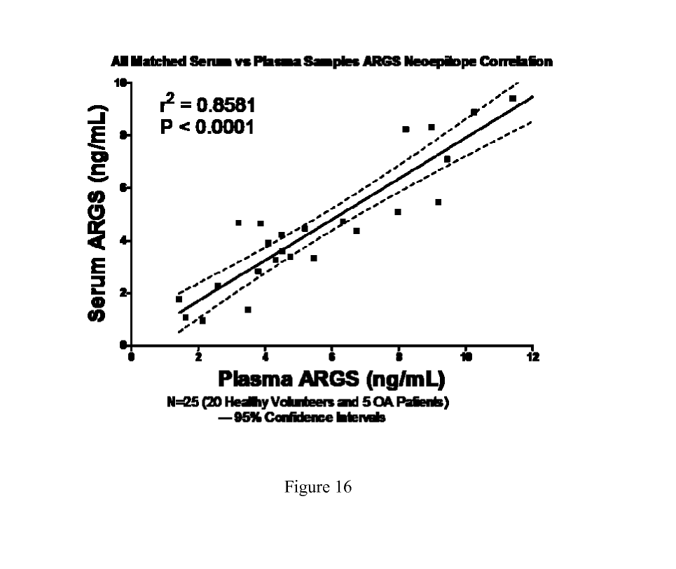

In addition, ARGS levels in healthy donor and OA donor matched serum and plasma show good correlation, r²=0.86, p<0.0001 (FIG. 16)

### Example 4

### Laboratory Methods

Quantification of ARGSVIL Neoepitope in human serum, plasma, synovial fluid and urine by msd immunoassay

This example describes an analytical method for the measurement of ARGSVIL neoepitope in human serum, plasma, synovial fluid and urine samples. Normal human serum, plasma and urine, and RA synovial fluid depleted of endogenous ARGSVIL neoepitope and aggrecan were as the assay calibrator matrix.

Materials/Equipment

Where indicated, equivalent equipment and supplies may be substituted for those listed.

| Equipment and Supplies | Manufacturer |
|---|---|
| Pipettes - Manual and repeaters | Gilson, Biohit and Eppendorf, |
| | or similar |
| MSD Sector Imager 6000 | Meso Scale Discovery (MSD) |
| MSD Standard Bind Plates | MSD (L11XA-3) |
| 0.5 mL and 2 mL Eppendorf tubes | Sarstedt (72.730.006 and 72.694.007 |
| | respectively), or similar |
| Plate sealers | In-house stores item (AH0045/1), |
| | or similar |
| Plate washer | Molecular Devices Skan Washer 300, |
| | or similar |
| Plate shaker | Delfia, or similar |

Reagents

| Reagent | Source (Catalogue Number) |
|---|---|
| 1M Hepes | Sigma (H0887) |
| TritonX-100 | Sigma (T8787) |
| MQ water. | in-house reagent |
| PBS | Sigma (14190-094) |
| Tween 20 | Sigma (P9416) |
| Mouse monoclonal anti-human | Invitrogen (AHP0022) |
| aggrecan antibody | |
| MSD Blocker A | MSD (R93BA-4) |
| MSD cytokine assay diluent | MSD (R51BB-2) |
| ARGSVIL neoepitope and aggrecan | In-house reagent |
| depleted serum, plasma, synovial | |
| fluid and urine | |
| Recombinant human aggrecan | R&D Systems (1220-PG) |
| G1-IGD-G2 domains | |
| ADAMTS5 enzyme | In-house reagent (GRITS 25511) |
| Anti-OA-1 neoepitope antibody | In-house reagent |
| MSD antibody diluent | MSD (R50AA-2) |
| Read Buffer T with surfactant | MSD (R92TC-1) |

Reagent preparation 25 mM Hepes and 0.015% Triton X-100 (Coating Buffer)

2.5 mL of 1M Hepes and 1504, of 10% Triton X-100 are added to 97.35 mL of MQ water. The solution was allowed to mix for 30 minutes and the solution filtered with a 0.2 μm filter before use. The solution can be stored at 2-8° C. for up to 6 months. PBS with 0.05% Tween 20 (Wash Buffer)

To generate 1L of wash buffer 500 μL Tween 20 was added to 1L of MQ water. The solution was gently inverted several times to ensure that the solution was mixed. Excess solution should be stored at room temperature for no longer than one month.

SulfoTAG Label of anti-OA-1 Ab according to MSD manufacturer's instructions.

Preparation of ARGSVIL Neoepitope Reference Standard:

2.254, of 1.2 μM ADAMTS-5 was incubated with 50 μL of 1 mg/mL Aggrecan (dissolved in 1×Biacore Buffer w/o BSA (pH 7.4) (10 mM HEPES, 1mM CaCl2, 150 mM NaCl2, 0.05% NP-40, 1 μM ZnCl2)) for 4 days at 4° C.

Sample Storage and Preparation

For long term storage, the test samples were kept at −80° C. The samples are thawed at room temperature and vortexed prior to analysis.

Procedure

1 μL of 25 μg/mL mouse monoclonal anti-human aggrecan antibody in coating buffer was spot coated onto a standard bind MSD plate:

An appropriate volume of mouse monoclonal anti-human aggrecan antibody at 25 μg/ml in coating buffer was prepared and dispensed to each well in column one of a Nunc V-bottom polypropylene 96-well plate. For example, to spot 1 MSD assay plate add 250 of 25 μg/mL mouse monoclonal anti-human aggrecan antibody in coating buffer to each well of column one (an overall volume of 10 μL per well was recommended regardless of the number of plates being coated).

The Mosquito HTS liquid handler was used to spot wells of MSD plates. The Mosquito MSD spotting method was used to transfer 1 μL of mouse monoclonal anti-human aggrecan antibody (25 μg/mL solution) to each well of a MSD standard-bind assay plate.

The spotted MSD plates were allowed to dry uncovered at RT for three days. Once dry, plates were stacked and the top plate sealed with an adhesive plate sealer. Coated plates should be batch tested and if successful, remaining plates can be stored for up to one month at 2-8° C.

On day of assay 3% blocker A in PBS (0.5 g blocker A+16.7 mL PBS (for 1 plate)) was prepared. The plate was washed 3×with 175 μL per well of PBS with 0.05% Tween 20 using a plate washer and the plate blotted dry with paper towels. 150 μL of 3% blocker A was added to each well of the plate and incubated 1 hr at room temperature on a shaker set at 600 rpm. The plate was washed 3× with 175 μL per well of PBS with 0.05% Tween 20 using a plate washer and the plate blotted dry with paper towels. 25 μL of MSD human serum cytokine assay diluent was added to each well of the plate and incubated 30 minutes at room temperature on a shaker set at 600 rpm

Preparations of an aggrecan neoepitope standard curve in matrix:

The standard curve was made as follows:

| | | | Stock |
|---|---|---|---|
| | Volume of | Volume and | concentration |
| Stock solution | matrix | source | (μg/mL) |
| Aggrecan | 90 μL | 10 μL of | 100 |
| neoepitope stock A | | 1 mg/mL stock | |
| | | aggrecan neoepitope | |

Preparation of Aggrecan Neoepitope Reference Standards

| | Volume of | Volume and | Concentration |
|---|---|---|---|
| Identification | matrix | source | (ng/mL) |
| Std 1 | 990 μL | 10 μL of | 1000.00 |
| | | Aggrecan | |
| | | neoepitope | |
| | | stock A | |
| Std 2 | 300 μL | 300 μL of Std 1 | 500.00 |
| Std 3 | 300 μL | 300 μL of Std 2 | 250.00 |
| Std 4 | 300 μL | 300 μL of Std 3 | 125.00 |
| Std 5 | 300 μL | 300 μL of Std 4 | 62.50 |
| Std 6 | 300 μL | 300 μL of Std 5 | 31.25 |
| Std 7 | 300 μL | 300 μL of Std 6 | 15.62 |
| Std 8 | 300 μL | 300 μL of Std 7 | 7.81 |
| Std 9 | 300 μL | 300 μL of Std 8 | 3.91 |
| Std 10 | 300 μL | 300 μL of Std 9 | 1.95 |
| Std 11 | 300 μL | 300 μL of Std 10 | 0.98 |
| Std 12 | 300 μL | — | 0 |
| (negative | | | |
| control (NC)) | | | |

25 μL of standards/samples was added to the plate and incubated for 2 hrs at room temperature on a shaker set at 600 rpm. A directly labelled anti-OA-1 detection antibody solution was prepared at 2 μg/mL (4.34 OA-1 stock (1.4 mg/mL)+2995.7 μL antibody diluent (for 1 plate)). The plate was washed 3× with 175 μL per well of PBS with 0.05% Tween 20 using a plate washer and the plate blotted dry with paper towels. 25 μL of detection antibody was added to the plate and incubated for 2 hr at room temperature on a shaker set at 600 rpm.

2× MSD Read Buffer T solution was prepared (8 mL 4×MSD read buffer T+8 mL water (for 1 plate)). The plate was washed 3× with 175 μL per well of PBS with 0.05% Tween 20 using a plate washer and the plate blotted dry with paper towels. 150 μL per well of 2× MSD Read Buffer T solution was added (taking care not to create any bubbles in the wells). The MSD assay plate was read immediately (within 15 minutes of read buffer addition) using the MSD Sector Imager 6000.

Example plate map

| | 1 | 2 | 3 | 4 | 5 | 6 | 7 | 8 | 9 | 10 | 11 | 12 |
|---|---|---|---|---|---|---|---|---|---|---|---|---|
| A | Std 1 | Std 2 | Std 3 | Std 4 | Std 5 | Std 6 | Std 7 | Std 8 | Std 9 | Std 10 | Std 11 | NC |
| B | | | | | | | | | | | | |

| | | | | |
|---|---|---|---|---|
| C | QC 2 | QC 3 | QC 4 | Sample 1 |

| | | | | | | |
|---|---|---|---|---|---|---|
| D | Sample 2 | Sample 3 | Sample 4 | Sample 5 | Sample 6 | Sample 7 |
| E | Sample 8 | Sample 9 | Sample 10 | Sample 11 | Sample 12 | Sample 13 |
| F | Sample 14 | Sample 15 | Sample 16 | Sample 17 | Sample 18 | Sample 19 |
| G | Sample 20 | Sample 21 | Sample 22 | Sample 23 | Sample 24 | Sample 25 |

| | | | | |
|---|---|---|---|---|
| H | Sample 26 | QC 2 | QC 3 | QC 4 |

1. A method for identifying a patient as a candidate for treatment with an aggrecanase inhibitor comprising:
isolating a biological sample from a patient; and
detecting in the sample the presence or absence of at least one aggrecan degradation product; wherein the presence of at least one aggrecan degradation product in the biological sample indicates that the patient is a good candidate for treatment.

2. The method of claim 1, wherein aggrecanase inhibitor inhibits the activity of an aggrecanse selected from the group consisting of ADAMTS1, ADAMTS4, ADAMTS5, ADAMTS9, and ADAMTS15.

3. The method of claim 1, wherein the aggrecanase inhibitor inhibits the activity of ADAMTS4 or ADAMTS5.

4. The method of claim 1, wherein the aggrecanase inhibitor is an antibody or a fragment thereof

5. The method of claim 1, wherein the at least one aggrecan degradation product comprises the neoepitope ARGSVIL.

6. The method of claim 1, wherein the patient is suffering from a disease or condition selected from the group consisting of chronic pain, neuropathic pain, postoperative pain, rheumatoid arthritis, osteoarthritis, neuralgia, neuropathies, algesia, nerve injury, ischaemia, neurodegeneration, cartilage degeneration, stroke, incontinence, inflammatory disorders, irritable bowel syndrome, periodontal disease, aberrant angiogenesis, tumor invasion and metastasis, corneal ulceration, complications of diabetes, psoriatic arthritis, inflammatory arthritis and chronic and/or acute kidney disease.

7. The method of claim 5, wherein the aggrecan degradation product is detected using an antibody or a fragment thereof

8. The method of claim 7, wherein the antibody or a fragment thereof used to detect the aggrecan degradation product is OA-1.

9. The method of claim 6, wherein the biological sample is human serum and the aggrecan degradation product comprising the neoepitope ARGSVIL is present at a concentration of at least about 6 ng/ml.

10. The method of claim 6, wherein the biological sample is human plasma and the aggrecan degradation product comprising the neoepitope ARGSVIL is present at a concentration of at least about 10 ng/ml.

11. The method of claim 6, wherein the biological sample is urine and the aggrecan degradation product comprising the neoepitope ARGSVIL is present at a concentration of at least about 5 ng/ml.

12. A method of evaluating the effectiveness of an aggrecanase inhibitor comprising
obtaining a first measurement of an aggrecan degradation product in a patient;
administering an aggrecanase inhibitor to the patient;
obtaining a second measurement of the aggrecan degradation product in the patient after administration of the aggrecanase inhibitor; and
comparing the first measurement to the second measurement; wherein an inhibition of aggrecanase activity is indicated when the second measurement of the aggrecan degradation product is less than the first measurement of the aggrecan degradation product.

13. The method of claim 12, wherein aggrecanase inhibitor inhibits the activity of an aggrecanse or MMP selected from the group consisting of ADAMTS1, ADAMTS4, ADAMTS5, ADAMTS9, and ADAMTS15.

14. The method of claim 12, wherein the aggrecanase inhibitor inhibits the activity of ADAMTS4 or ADAMTS5.

15. The method of claim 12, wherein the aggrecanase inhibitor is an antibody or a fragment thereof.

16. The method of claim 12, wherein the at least one aggrecan degradation product comprises the neoepitope ARGSVIL.

17. The method of claim 12, wherein the patient is suffering from a disease or condition selected from the group consisting of chronic pain, neuropathic pain, postoperative pain, rheumatoid arthritis, osteoarthritis, neuralgia, neuropathies, algesia, nerve injury, ischaemia, neurodegeneration, cartilage degeneration, stroke, incontinence, inflammatory disorders, irritable bowel syndrome, periodontal disease, aberrant angiogenesis, tumor invasion and metastasis, corneal ulceration, and complications of diabetes.

18. The method of claim 16, wherein the aggrecan degradation product is detected using an antibody or a fragment thereof.

19. The method of claim 18, wherein the antibody or a fragment thereof used to detect the aggrecan degradation product is OA-1.

20. The method of claim 15, wherein the antibody comprises a heavy chain comprising CDRH1, CDRH 2 and CDRH3 and a light chain comprising CDRL1, CDRL 2 and CDRL 3, wherein the complementarity determining regions (CDRs) of the heavy chain are selected from the group of:
a) CDRH1 having at least about 80% sequence identity to amino acid sequence DAWMD;
b) CDRH2 having at least about 70% sequence identity to amino acid sequence EIRHKANDHAIFYXESVKG; and
c) CDRH3 having at least about 70% sequence identity to amino acid sequence TYYYGSSYGYCDV or PFAY; and
the complementarity determining regions of the light chain are selected from the group of:
d) CDRL1 having at least about 70% sequence identity to amino acid sequence KASQSVGTTIV or RTSENIYSYLA;
e) CDRL2 having at least about 70% sequence identity to amino acid sequence NAKTLAE or SASNRXT; and
f) CDRL3 having at least about 70% sequence identity to amino acid sequence QQYSSYPFT or QHHYGTPWT.

21. (canceled)

22. (canceled)
